(19) United States
(12) Patent Application Publication
(10) Pub. No.: US 2023/0285615 A1
(43) Pub. Date: Sep. 14, 2023

(54) ENDOSCOPE STERILIZATION DEVICE AND ENDOSCOPE STERILIZATION SYSTEM

(71) Applicant: FUJIFILM Corporation, Tokyo (JP)

(72) Inventors: Kojiro KOTANI, Kanagawa (JP); Yutaka SENDA, Kanagawa (JP); Shozo IYAMA, Kanagawa (JP)

(73) Assignee: FUJIFILM Corporation, Tokyo (JP)

(21) Appl. No.: 18/319,453

(22) Filed: May 17, 2023

Related U.S. Application Data
(63) Continuation of application No. PCT/JP2021/040074, filed on Oct. 29, 2021.

(30) Foreign Application Priority Data
Nov. 19, 2020 (JP) 2020-192469

(51) Int. Cl. A61L 2/10; A61L 2/24; A61B 1/12

(52) U.S. Cl. CPC A61L 2/10; A61L 2/24; A61B 1/121; A61L 2202/24; A61L 2202/11; A61L 2202/122; A61L 2202/121; A61L 2202/16

(57) ABSTRACT

An endoscope sterilization device irradiates a distal end portion of an endoscope with light for sterilization. The endoscope sterilization device includes a housing that has a bottomed tubular shape including an opening portion, a peripheral surface portion, and a bottom surface portion and into which at least a portion of the distal end portion is insertable through the opening portion and a sterilization light source that is provided in the housing and that irradiates the distal end portion inserted into the housing with the light for sterilization.

### CROSS-REFERENCE TO RELATED APPLICATIONS

This application is a Continuation of PCT International Application No. PCT/JP2021/040074 filed on 29 Oct. 2021, which claims priority under 35 U.S.C § 119(a) to Japanese Patent Application No. 2020-192469 filed on 19 Nov. 2020. The above application is hereby expressly incorporated by reference, in its entirety, into the present application.

### BACKGROUND OF THE INVENTION

### 1. Field of the Invention

The present invention relates to an endoscope sterilization device and an endoscope sterilization system.

### 2. Description of the Related Art

In the medical field, endoscopy is performed in which an insertion portion of an endoscope is inserted into a subject and then observation is performed. It is known that such an endoscope is hung and held by means of a hanger in a standby state before observation or the like. In addition, in many cases, a hanger that holds an endoscope is provided on an endoscope cart on which the endoscope, a processor device, a light source device, and the like are placed to be transported.

Described in JP1993-95888A (JP-H5-95888A) is an endoscope cart provided with a hanger that holds an endoscope. The endoscope cart includes a hanger cover that covers the hanger, a heating unit that increases the temperature of the vicinity of the endoscope held by the hanger, and the like. The above-described configuration is to keep the endoscope clean. In addition, in the case of the endoscope cart described in JP1993-95888A (JP-H5-95888A), a sterilization lamp is disposed in the hanger cover so that growth of bacteria in the hanger cover is suppressed.

In the case of an endoscope cart described in JP1998-262902A (JP-H10-262902A), a sanitary case is attached to a side surface of an endoscope cart main body. A hanger, a support that supports the hanger, and a sterilization lamp are provided in the sanitary case. The support includes an irradiation window, and the sterilization lamp is attached inside the support. Through the irradiation window, the endoscope hung on the hanger is irradiated with ultraviolet rays from the sterilization lamp turned on, so that the endoscope is sterilized.

### SUMMARY OF THE INVENTION

In the medical field, since it is desired to take additional measures to prevent an infectious disease and an insertion portion of an endoscope is inserted into a subject to perform observation, sterilization with respect to bacteria such as viruses is important. Particularly, performing the sterilization immediately before the insertion portion is inserted into the subject is required. However, even in a case where a sterilization lamp is provided in a hanger cover as in the case of the endoscope cart described in JP1993-95888A (JP-H5-95888A), a sterilization effect is exhibited mainly for the hanger cover with sterilization of the insertion portion of the endoscope being not taken into account.

Meanwhile, in the case of the endoscope cart described in JP1998-262902A (JP-H10-262902A), the sterilization lamp is attached inside the support that supports the hanger. However, a position where the ultraviolet rays collide with the endoscope and the amount of irradiation vary depending on the way in which the endoscope is hung on the hanger and thus a high sterilization effect cannot be achieved for the insertion portion of the endoscope. That is, in the case of the endoscope carts described in JP1993-95888A (JP-H5-95888A) and JP1998-262902A (JP-H10-262902A), sterilizing the insertion portion immediately before the insertion portion is inserted into the subject has not been taken into account.

An object of the present invention is to provide an endoscope sterilization device and an endoscope sterilization system with which it is possible to achieve a high sterilization effect with respect to an insertion portion of an endoscope.

An endoscope sterilization device according to an aspect of the present invention is an endoscope sterilization device that irradiates an insertion portion of an endoscope with light for sterilization and that includes an accommodation portion and a sterilization light source. The accommodation portion has a bottomed tubular shape including an opening portion, a peripheral surface portion, and a bottom surface portion and at least a portion of the insertion portion is insertable into the accommodation portion through the opening portion. The sterilization light source is provided in the accommodation portion and irradiates the insertion portion inserted into the accommodation portion with the light for sterilization.

It is preferable that the sterilization light source is provided on the bottom surface portion or on the peripheral surface portion. It is preferable that a reflective material is provided on at least one of the bottom surface portion or the peripheral surface portion.

It is preferable that the light with which the sterilization light source performs the irradiation is ultraviolet rays of which a central wavelength is 222 nm. It is preferable that the sterilization light source is an excimer lamp or an LED.

It is preferable that the endoscope sterilization device further includes a lid member that constitutes at least a portion of the bottom surface portion or the peripheral surface portion and the lid member is movable between a closing position at which an inside of the accommodation portion is covered and an opening position at which the inside of the accommodation portion is exposed. It is preferable that the lid member is attachably and detachably separated from the accommodation portion.

It is preferable that the endoscope sterilization device further includes an insertion amount detection unit that detects an amount of insertion of the insertion portion into the accommodation portion. It is preferable that the endoscope sterilization device further includes an insertion portion holding portion that holds the insertion portion inserted into the accommodation portion.

An endoscope sterilization system according to another aspect of the present invention is an endoscope sterilization system in which an insertion portion of an endoscope is irradiated with light for sterilization, the endoscope sterilization system including: an endoscope holding device that is provided with a hanger holding an operation portion of the endoscope, the operation portion being consecutively connected to the insertion portion; and the above-described endoscope sterilization device.

It is preferable that the endoscope holding device is an endoscope stand or an endoscope cart.

It is preferable that the opening portion is open in a direction intersecting a hanging direction in which the insertion portion hangs down because of a weight thereof in a case where the operation portion is held by the hanger.

It is preferable that the endoscope sterilization system further includes an attachment and detachment mechanism that makes the endoscope sterilization device and the endoscope holding device attachable to each other and detachable from each other.

An endoscope sterilization system according to still another aspect of the present invention is an endoscope sterilization system in which an insertion portion of an endoscope is irradiated with light for sterilization, the endoscope sterilization system including an endoscope holding device and an endoscope sterilization device. The endoscope holding device is provided with a hanger holding an operation portion of the endoscope, the operation portion being consecutively connected to the insertion portion. The endoscope sterilization device includes a gutter-shaped accommodation portion and a sterilization light source, the accommodation portion being composed of at least a peripheral surface portion that faces an outer peripheral surface of the insertion portion hanging down because of a weight thereof in a case where the operation portion is held by the hanger and the sterilization light source irradiating the insertion portion accommodated in the accommodation portion with the light for sterilization.

An endoscope sterilization device according to still another aspect of the present invention is an endoscope sterilization device that irradiates an insertion portion of an endoscope with light for sterilization and that includes an insertion part and a sterilization light source. The insertion part includes a peripheral surface portion facing the insertion portion and the insertion portion is insertable into the insertion part. The sterilization light source irradiates the insertion portion inserted into the insertion part with the light for sterilization.

An endoscope sterilization system according to still another aspect of the present invention is an endoscope sterilization system in which an insertion portion of an endoscope is irradiated with light for sterilization, the endoscope sterilization system including an endoscope holding device, the above-described endoscope sterilization device, and a slide mechanism. The endoscope holding device is provided with a hanger holding an operation portion of the endoscope, the operation portion being consecutively connected to the insertion portion. The slide mechanism holds the endoscope sterilization device to be slidable along a hanging direction in which the insertion portion hangs down because of a weight thereof in a case where the operation portion is held by the hanger.

It is preferable that the slide mechanism includes a speed attenuation mechanism that attenuates a speed of movement of the endoscope sterilization device. It is preferable that the endoscope holding device is an endoscope stand or an endoscope cart.

It is preferable that the endoscope sterilization device further includes a grip portion that is integrally provided with the insertion part. It is preferable that the endoscope sterilization device further includes a cover member that is provided to be attachable to and detachable from the insertion part, and, in a case where the cover member and the insertion part are integrated with each other, the cover member and the insertion part constitute an accommodation portion that has a bottomed tubular shape including an opening portion, a peripheral surface portion, and a bottom surface portion and into which at least a portion of the insertion portion is insertable through the opening portion.

According to the aspects of the present invention, it is possible to achieve a high sterilization effect with respect to an insertion portion of an endoscope.

### BRIEF DESCRIPTION OF THE DRAWINGS

FIG. 1 is a perspective view of an endoscope system and an endoscope sterilization system.

FIG. 2 is a perspective view of an endoscope sterilization device.

FIG. 3 is a cross-sectional view of a main part of the endoscope sterilization device.

FIG. 4 is a block diagram showing the outline of an electrical configuration of the endoscope sterilization device.

FIG. 5 is a perspective view of an endoscope sterilization system according to a second embodiment.

FIG. 6 is a cross-sectional view of a main part of an endoscope sterilization device according to the second embodiment.

FIG. 7 is a perspective view of an endoscope sterilization device according to a third embodiment.

FIG. 8 is a schematic view showing the configurations of an insertion portion holding portion and an insertion amount detection unit of an endoscope sterilization device and an endoscope sterilization system of a first modification example.

FIG. 9 is a perspective view of an endoscope sterilization system of a second modification example.

FIG. 10 is a perspective view showing a state in which a portion of an endoscope sterilization device is removed in the endoscope sterilization system of the second modification example.

FIG. 11 is a cross-sectional view of a main part of the endoscope sterilization system of the second modification example.

FIG. 12 is a perspective view of an endoscope sterilization system according to a fourth embodiment.

FIG. 13 is a perspective view of an endoscope sterilization device according to the fourth embodiment.

FIG. 14 is a cross-sectional view of a main part of the endoscope sterilization device according to the fourth embodiment.

FIG. 15 is a perspective view of an endoscope sterilization system according to a fifth embodiment.

FIG. 16 is a perspective view of an endoscope sterilization device to the fifth embodiment.

FIG. 17 is a perspective view of an endoscope sterilization device according to a sixth embodiment.

FIG. 18 is a perspective view showing a state in which a cover member is separated from a sterilization device main body in the endoscope sterilization device according to the sixth embodiment.

FIGS. 19A and 19B are perspective views of an endoscope sterilization device of a third modification example, where FIG. 19A is a perspective view showing a mode used for sterilization of an endoscope and FIG. 19B is a perspective view showing a mode used for sterilization of an instrument for an endoscope.

FIG. 20 is a perspective view showing an example in which the endoscope sterilization device according to the third modification example is used for sterilization of a processor device.

FIG. 21 is a perspective view of an endoscope sterilization system according to a seventh embodiment.

FIG. 22 is a side view showing the endoscope sterilization system according to the seventh embodiment.

FIG. 23 is a perspective view of an endoscope sterilization device according to the seventh embodiment as seen from a rear surface side.

FIG. 24 is a perspective view of an endoscope sterilization device of a fourth modification example as seen from a rear surface side.

### DESCRIPTION OF THE PREFERRED EMBODIMENTS

### First Embodiment

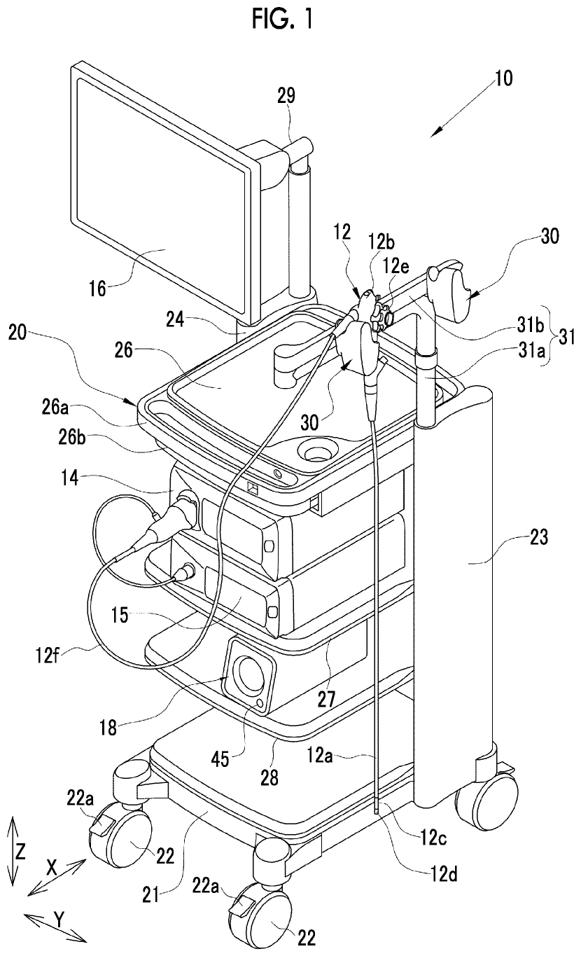

As shown in FIG. 1, an endoscope system 10 includes an endoscope 12, a light source device 14, a processor device 15, a display 16, a keyboard (not shown), an endoscope sterilization device 18, and an endoscope cart 20 on which these devices are placed to be transported. The endoscope cart 20 constitutes an endoscope holding device in the scope of the claims. The endoscope 12 images an observation target. The light source device 14 emits illumination light with which the observation target is irradiated. The processor device 15 controls the endoscope system 10. The display 16 is a display unit that displays an observation image or the like based on an endoscopic image. The keyboard is an input device used to input settings or the like with respect to the processor device 15.

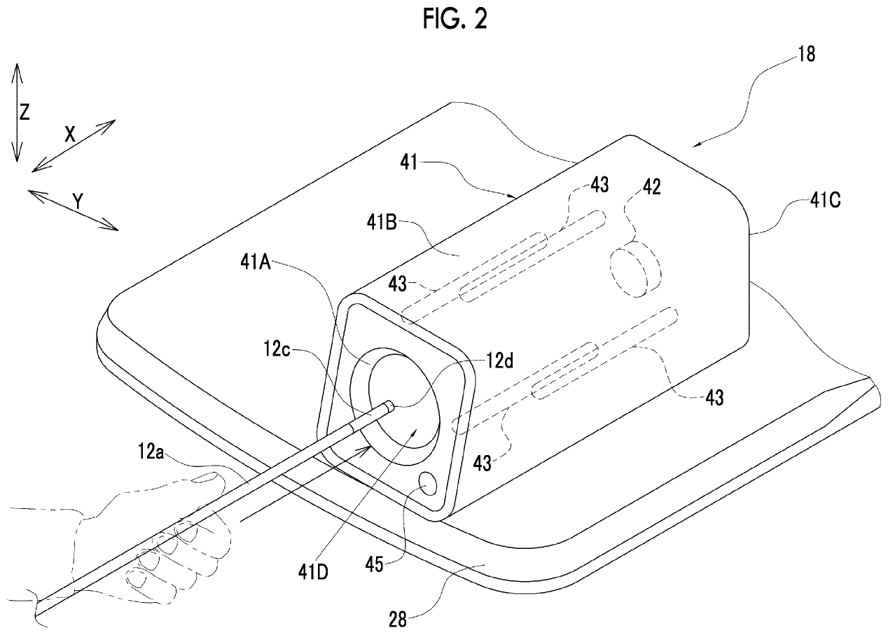

The endoscope 12 includes a soft portion 12a, an operation portion 12b consecutively connected to a proximal end portion of the soft portion 12a, a bendable portion 12c (refer to FIG. 2) provided on a distal end side of the soft portion 12a, and a distal end portion 12d (refer to FIG. 2). An insertion portion of the endoscope 12 is composed of the soft portion 12a, the bendable portion 12c, and the distal end portion 12d. In a case where an angle knob 12e of the operation portion 12b is operated, the bendable portion 12c is bent. As a result, the distal end portion 12d faces a desired direction. The endoscope 12 includes a cord 12f connected to the light source device 14 and the processor device 15, is optically connected to the light source device 14, and is electrically connected to the processor device 15.

The endoscope sterilization device 18 and the endoscope cart 20 constitute an endoscope sterilization system in the scope of the claims. The endoscope cart 20 includes a frame pedestal portion 21, four casters 22, columns 23 and 24, a top plate 26, shelf plates 27 and 28, a stand 29, and two hangers 30. The frame pedestal portion 21 is formed in an approximately rectangular shape and the casters 22 are provided at four corners thereof. The columns 23 and 24 are columnar members that are erected to extend upward from both of side end portions of the frame pedestal portion 21. The top plate 26 is formed in an approximately rectangular shape and is disposed at upper ends of the columns 23 and 24. Both of side end portions of the top plate 26 are fixed to inner sides of upper end portions of the columns 23 and 24.

The shelf plates 27 and 28 are each formed in an approximately rectangular shape and are fixed to inner sides of intermediate portions of the columns 23 and 24. The shelf plates 27 and 28 partition a space between the frame pedestal portion 21 and the top plate 26. Each caster 22 is provided with a stop lever 22a for restriction of rotation of the caster 22. The rotation of the caster 22 is restricted in a case where the stop lever 22a is in a lowered state and the rotation of the caster 22 is allowed in a case where the stop lever 22a is in a raised state.

The top plate 26 and the shelf plates 27 and 28 are panels with upper surfaces on which an instrument for an endoscope or the like is to be placed and in an example shown in FIG. 1, the processor device 15 and the light source device 14 are placed on an upper surface of the shelf plate 27 and the endoscope sterilization device 18 is placed on an upper surface of the shelf plate 28.

A front portion of the top plate 26 is provided with a handle 26a. A user can easily move the endoscope cart 20 while holding the handle 26a with a hand. A lower surface of the top plate 26 is provided with a drawer portion 26b. A keyboard or the like that is used for input to the processor device 15 or the like is stored in the drawer portion 26b. The display 16 is fixed to the stand 29 attached to an upper end of the column 24 on the left side, and is connected to the processor device 15 via a wiring line in the stand 29 and the column 24.

The hangers 30 are attached to an upper end of the column 23 on the right side via a hanger stand 31. The hanger stand 31 is composed of an L-shaped stand member 31a and a linear stand member 31b. One end portion of the stand member 31a is rotatably attached to the column 23. The hangers 30 are fixed to the stand member 31b. The hangers 30 hold the operation portion 12b of the endoscope 12 as will be described later. The stand member 31b is rotatably connected to the other end portion of the stand member 3 la. Accordingly, it is possible to adjust the positions of the hangers 30 and the endoscope 12 held by the hangers 30 by rotating the stand members 31a and 31b with respect to the column 23.

Each hanger 30 includes a notch or a groove, and the endoscope 12 can be hung on the hanger 30 with the operation portion 12b of the endoscope 12 being held at the notch or the groove. In a case where the operation portion 12b is held by the hanger 30, a hanging direction in which the soft portion 12a hangs down is parallel to a vertical direction Z, the soft portion 12a hanging down because of the weight thereof. The position of the hanger 30 is adjusted such that the distal end portion 12d does not come into contact with a floor surface in a case where the endoscope 12 is hung. The number of hangers 30 is not limited to two and an appropriate number of hangers 30 are provided in accordance with the purpose of use of the endoscope 12 and the dimensions of the endoscope cart 20 and the hanger stand 31.

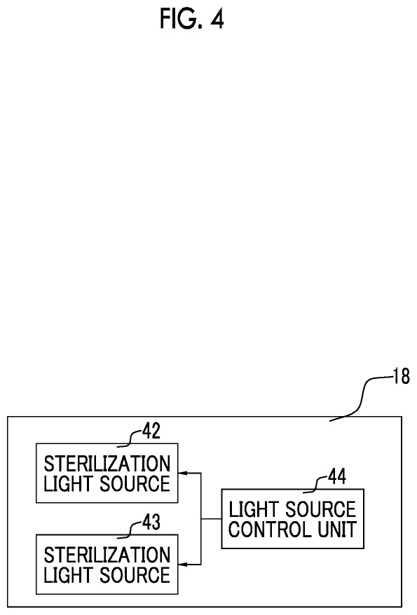

As shown in FIG. 2, the endoscope sterilization device 18 includes a housing 41 as an accommodation portion, sterilization light sources 42 and 43, a light source control unit 44 (refer to FIG. 4), and a power switch 45. The endoscope sterilization device 18 sterilizes at least a portion of the insertion portion of the endoscope 12. In the present embodiment, the endoscope sterilization device 18 mainly sterilizes the distal end portion 12d.

The housing 41 is formed in a bottomed tubular shape including an opening portion 41A, a peripheral surface portion 41B, and a rear surface portion 41C. The peripheral surface portion 41B is formed in a quadrangular tubular shape to constitute upper and lower surfaces and right and left surfaces of the housing 41. The rear surface portion 41C closes a rear end of the peripheral surface portion 41B. The rear surface portion 41C corresponds to a bottom surface portion in the scope of the claims.

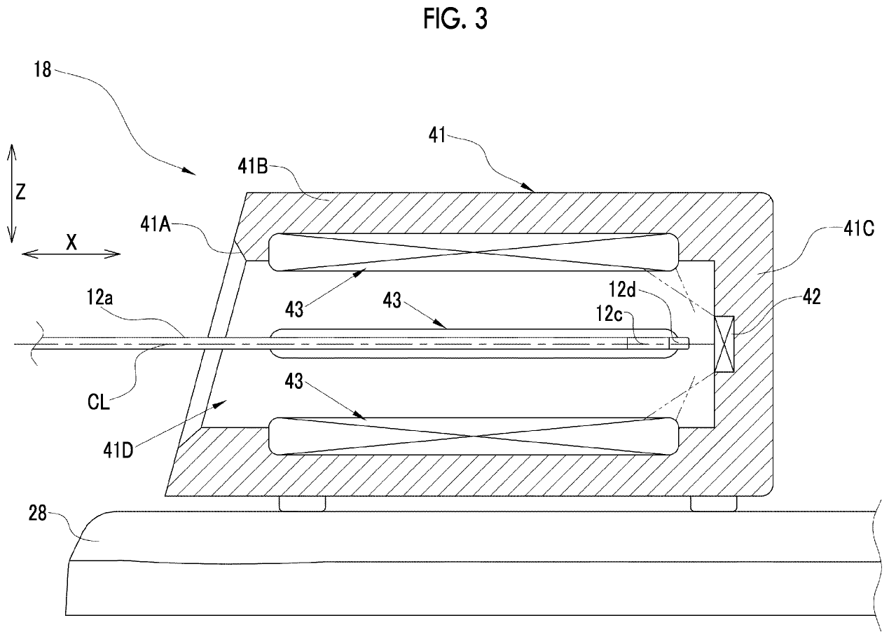

A space inside the housing 41 that is surrounded by the peripheral surface portion 41B and the rear surface portion 41C is a sterilization space 41D, which is a space in which irradiation with ultraviolet rays for sterilization (which will be described later) is performed. The sterilization space 41D is formed to have dimensions larger than at least the outer diameter and the axial length of the distal end portion 12d, and can accommodate the distal end portion 12d. The opening portion 41A is a circular through-hole that is positioned in a front surface of the peripheral surface portion 41B and that has an inner diameter larger than at least the outer diameter of the distal end portion 12d. A direction in which the opening portion 41A is open, that is, a direction parallel to a central axis CL (refer to FIG. 3) of the opening portion 41A intersects the vertical direction Z which is the direction in which the soft portion 12a hangs down. Note that the direction in which the opening portion 41A is open is appropriately changed depending on a direction in which the endoscope sterilization device 18 is disposed. However, in the present embodiment, for the sake of convenience related to description, a front-rear direction X of the endoscope cart 20 is the direction in which the opening portion 41A is open. Through the opening portion 41A, the distal end portion 12d which is at least a portion of the insertion portion can be inserted into the sterilization space 41D from the outside of the housing 41. The power switch 45 is provided in the vicinity of the opening portion 41A.

The sterilization light source 42 is provided on the rear surface portion 41C of the housing 41. As the sterilization light source 42, an ultraviolet ray source is used. The sterilization light source 42 is any of an excimer lamp or a light emitting diode (LED) for ultraviolet rays. In addition, the excimer lamp is a discharge lamp that emits light from an excimer formed of a rare gas atom or a rare gas atom and a halogen atom by using a feature that a large number of short-time dielectric barrier discharges occur and there are a two-layer cylindrical type and a flat plate type as the structure thereof. As the sterilization light source 42, for example, a flat plate type excimer lamp is used.

The excimer lamp preferably emits first ultraviolet rays having a peak wavelength of 222 nm (±10 nm) and a wavelength range of 200 nm to 280 nm, and more preferably emits the first ultraviolet rays having a wavelength range of 200 nm to 230 nm. The transmittance of the first ultraviolet rays with respect to a living body is lower than the transmittance of ultraviolet rays (specific ultraviolet rays) at a wavelength of 254 nm (at which the intensity of light is high) with respect to a living body. For example, the transmittance of the first ultraviolet rays with respect to a cornea of an eye (the ratio of light absorbed by the cornea to light incident on the cornea) is 0.01 and the transmittance of the specific ultraviolet rays with respect to the cornea is 30%. Therefore, the first ultraviolet rays have an ability for sterilization and inactivation of viruses, which is inherent in ultraviolet rays, while being safe for the skin and eyes of humans and animals.

It is preferable that the LED for ultraviolet rays emits second ultraviolet rays, which are deep ultraviolet rays having a wavelength range of 300 nm or less, as ultraviolet rays. Since the second ultraviolet rays have a function of destroying genetic information of a virus or a bacterium, the second ultraviolet rays have a sterilization effect on water, air, or the like. For example, the second ultraviolet rays have a high sterilization effect against the human coronavirus (HCoV-229E), as a virus. In addition, it is preferable that the LED for ultraviolet rays has a function such as a waterproofing function or a heat radiation function. The influence of the LED for ultraviolet rays on the environment is smaller than that of a mercury lamp.

The sterilization light source 42 is exposed at an innermost surface in the sterilization space 41D. The sterilization light source 42 includes an optical member (not shown) and diffuses ultraviolet rays to irradiate the sterilization space 41D with the ultraviolet rays. The ultraviolet rays can be diffused such that the range of irradiation covers at least all of the surfaces of the distal end portion 12d.

The sterilization light sources 43 are provided on the peripheral surface portion 41B of the housing 41. In the example shown in FIGS. 2 and 3, the sterilization light sources 43 are long sterilization light sources provided on upper and lower surfaces and right and left surfaces in the sterilization space 41D, and are disposed to be parallel with the front-rear direction X. Note that as the sterilization light sources 43, the same ultraviolet ray sources as the above-described sterilization light source 42 are used. However, it is preferable that cylindrical type excimer lamps are used as the sterilization light sources 43.

As shown in FIG. 4, the sterilization light sources 42 and 43 are controlled by the light source control unit 44. The sterilization light sources 42 and 43 that have started irradiation with ultraviolet rays by being controlled by the light source control unit 44 automatically stop, based on measurement performed by a timer built in the light source control unit 44, the irradiation with the ultraviolet rays in a case where a predetermined time (several seconds to several minutes) elapses. The way in which the sterilization light sources 42 and 43 are controlled by the light source control unit 44 is not limited thereto. For example, an optical or physical sensor that detects the state of insertion of the insertion portion into the housing 41 may be provided, so that the sterilization light sources 42 and 43 are controlled to be turned on so as to start the irradiation with the ultraviolet rays in a case where insertion of the insertion portion (any of the soft portion 12a, the bendable portion 12c, or the distal end portion 12d) into the housing 41 is detected and the sterilization light sources 42 and 43 are controlled to be turned off so as to stop the irradiation with the ultraviolet rays in a case where removal of the insertion portion from the housing 41 is detected.

Note that, the way in which the sterilization light source 42 is controlled by the light source control unit 44 is not limited to the above-described way. For example, the irradiation with ultraviolet rays may be stopped even before the predetermined time elapses in a case where a predetermined condition is satisfied. Regarding the predetermined condition herein, for example, an optical or physical sensor that detects the state of insertion of the insertion portion into the housing 41 is provided in the endoscope sterilization device 18 and the light source control unit 44 determines that the predetermined condition is satisfied and stops the irradiation with the ultraviolet rays in a case where the insertion portion is removed from the housing 41.

In addition, regarding the way in which the sterilization light source 42 is controlled by the light source control unit 44, in a case where the actual irradiation time is shorter than a setting time set in advance in a case where the irradiation with the ultraviolet rays is stopped once because the above-described predetermined condition is satisfied before the predetermined time elapses, control is performed such that a user is notified to that effect. Then, in a case where the predetermined condition is dissatisfied after the notification, the light source control unit 44 causes the sterilization light source 42 to restart the irradiation with the ultraviolet rays. The meaning of “a case where the predetermined condition is dissatisfied” is, for example, a case where it is detected that the insertion portion has been inserted into the housing 41 again. Note that as a method of notifying the user that the irradiation time is short, for example, a method of outputting a voice or changing what an indicator displays is used.

The action of the above-described configuration will be described. Before observation is performed by means of the endoscope 12, the operation portion 12b of the endoscope 12 after being cleaned is held by the hanger 30. In a case where the operation portion 12b is held by the hanger 30, the soft portion 12a enters a state of hanging down because of the weight thereof. A doctor or a medical staff, who is a user, operates the power switch 45 of the endoscope sterilization device 18 to turn on the power. Then, the distal end portion 12d is inserted into the housing 41 (that is, the sterilization space 41D) through the opening portion 41A. At least a portion of the insertion portion including the distal end portion 12d is irradiated with ultraviolet rays from the sterilization light sources 42 and 43. The distal end portion 12d is reliably sterilized by being irradiated with the ultraviolet rays, and thus a high sterilization effect can be achieved.

In addition, since ultraviolet rays having a wavelength range of 200 nm to 280 nm are used as the ultraviolet rays from the sterilization light sources 42 and 43 with which at least a portion of the insertion portion is irradiated, sterilization can be performed more reliably.

### Second Embodiment

In the first embodiment described above, an endoscope sterilization device that is placed on the endoscope cart 20 has been described as an example of the endoscope sterilization device 18. However, the present invention is not limited thereto and in a second embodiment which will be described below, an endoscope sterilization device that is attached to the endoscope cart 20 will be described. The configuration of the endoscope cart 20 is the same as that of the first embodiment and thus the description thereof will be omitted.

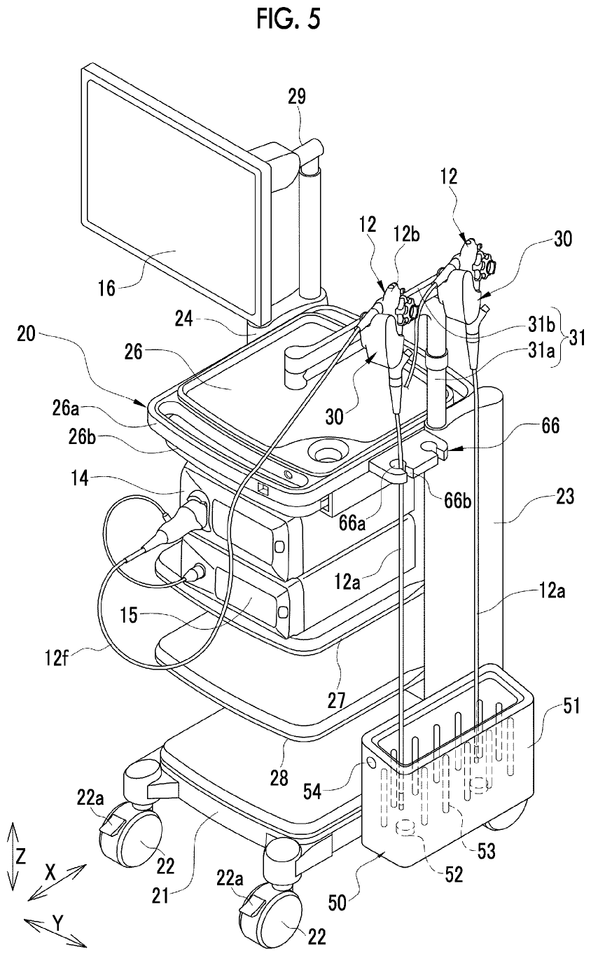

As shown in FIG. 5, an endoscope sterilization device 50 is attached to the column 23 of the endoscope cart 20. The endoscope sterilization device 50 and the endoscope cart 20 constitute an endoscope sterilization system in the scope of the claims. The endoscope sterilization device 50 is disposed below the hangers 30. That is, the endoscope sterilization device 50 is positioned on a distal end side in the hanging direction (that is, the vertical direction Z) in which the soft portion 12a hangs down because of the weight thereof in a case where the operation portion 12b is held by the hanger 30 and in a case where the operation portion 12b of the endoscope 12 is held by the hanger 30, the distal end portion 12d is inserted into the endoscope sterilization device 50.

The endoscope sterilization device 50 includes a housing 51 as an accommodation portion, sterilization light sources 52 and 53, a light source control unit (not shown), and a power switch 54. The endoscope sterilization device 50 sterilizes at least a portion of the insertion portion of the endoscope 12. In the present embodiment, the endoscope sterilization device 50 mainly sterilizes the distal end portion 12d. In addition, the housing 51 includes a space in which a plurality of the distal end portions 12d can be accommodated and in an example shown in FIG. 5, the endoscope sterilization device 50 sterilizes two distal end portions 12d at the same time.

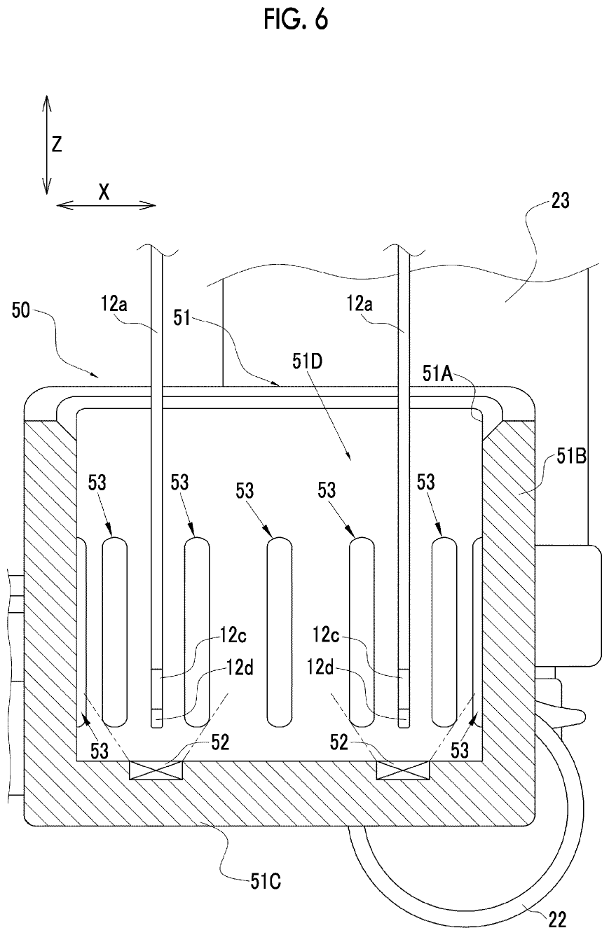

As shown in FIG. 6, the housing 51 is formed in a bottomed tubular shape including an opening portion 51A, a peripheral surface portion 51B, and a bottom surface portion 51C. The peripheral surface portion 51B is formed in a quadrangular tubular shape to constitute front and rear surfaces and right and left surfaces of the housing 51. The bottom surface portion 51C closes a lower end of the peripheral surface portion 51B.

A space inside the housing 51 that is surrounded by the peripheral surface portion 51B and the bottom surface portion 51C is a sterilization space 51D, which is a space in which irradiation with ultraviolet rays for sterilization is performed. The sterilization space 51D is formed to have dimensions larger than at least the outer diameter and the axial length of the distal end portion 12d, and can accommodate the plurality of distal end portions 12d. The opening portion 51A is a rectangular through-hole that is positioned in an upper surface of the peripheral surface portion 51B and through which the plurality of distal end portions 12d pass. A direction in which the opening portion 51A is open is parallel to the vertical direction Z, which is the hanging direction of the soft portion 12a. Accordingly, the distal end portions 12d can be inserted into the sterilization space 51D from the outside of the housing 51 through the opening portion 51A. The power switch 54 is provided in the vicinity of the opening portion 51A.

The sterilization light sources 52 are provided on the bottom surface portion 51C of the housing 51. As the sterilization light sources 52, ultraviolet ray sources are used. The sterilization light sources 52 are exposed at a lowermost surface in the sterilization space 51D. The sterilization light sources 52 each include an optical member (not shown) and diffuse ultraviolet rays to irradiate the sterilization space 51D with the ultraviolet rays. The ultraviolet rays can be diffused such that the range of irradiation covers at least all of the surfaces of the distal end portion 12d. In an example shown in FIG. 6, two sterilization light sources 52 are provided in accordance with the positions of insertion of the distal end portions 12d.

The sterilization light sources 53 are provided on the peripheral surface portion 51B of the housing 51. The sterilization light sources 53 are long sterilization light sources provided on front and rear surfaces and right and left surfaces in the sterilization space 51D, and are disposed to be parallel with the vertical direction Z. Note that as the sterilization light sources 52 and 53, the same ultraviolet ray sources as the above-described sterilization light sources 42 and 43 in the first embodiment are used. However, it is preferable that flat plate-type excimer lamps are used as the sterilization light sources 52 and cylindrical type excimer lamps are used as the sterilization light sources 53.

The sterilization light sources 52 and 53 are controlled by the light source control unit. The light source control unit of the present embodiment performs the same control as the light source control unit 44 of the first embodiment, and the sterilization light sources 52 and 53 automatically stop irradiation with ultraviolet rays in a case where a predetermined time (several seconds to several minutes) elapses. The way in which the sterilization light sources 52 and 53 are controlled by the light source control unit is not limited thereto. For example, a motion sensor or the like that detects the presence or absence of a human in the vicinity of the housing 51 may be provided in the endoscope sterilization device 50 or the endoscope cart 20, so that the sterilization light sources 52 and 53 are controlled to start the irradiation with the ultraviolet rays in a case where it is detected that a human is present in the vicinity of the housing 51 and the sterilization light sources 52 and 53 are controlled to stop the irradiation with the ultraviolet rays in a case where it is detected that a human is not present in the vicinity of the housing 51.

In addition, regarding the way in which the sterilization light sources 52 and 53 are controlled by the light source control unit, the irradiation with the ultraviolet rays is stopped in a case where a predetermined condition is satisfied even before a predetermined time elapses. Alternately, in a case where the actual irradiation time is shorter than a setting time set in advance in a case where the irradiation with the ultraviolet rays is stopped once because the predetermined condition is satisfied before the predetermined time elapses, control is performed such that a user is notified to that effect or the like. Note that, regarding a case where the predetermined condition is satisfied, the light source control unit may determine that the predetermined condition is satisfied and stop the irradiation with the ultraviolet rays in a case where the insertion portion is removed from the housing 51. Alternately, a motion sensor may be provided in the endoscope sterilization device 50 or the endoscope cart 20 and the light source control unit may determine that the predetermined condition is satisfied and stop the irradiation with the ultraviolet rays in a case where it is detected that a human is not present in the vicinity of the housing 51.

The action of the above-described configuration will be described. In a case where the operation portion 12b of the endoscope 12 is held by the hanger 30 before observation is performed by means of the endoscope 12, as described above, the endoscope sterilization device 50 is disposed in the hanging direction in which the soft portion 12a hangs down because of the weight thereof. Accordingly, in a case where the operation portion 12b is held by the hanger 30, the endoscope sterilization device 50 is positioned in the vicinity of the distal end portion 12d. Therefore, a doctor or a medical staff, who is a user, can easily insert the distal end portion 12d into the housing 51, (that is, the sterilization space 51D) through the opening portion 51A. In addition, the doctor or the medical staff operates the power switch 54 of the endoscope sterilization device 50 to turn on the power. As with the above-described first embodiment, the distal end portion 12d is reliably sterilized by being irradiated with the ultraviolet rays, and thus a high sterilization effect can be achieved.

### Third Embodiment

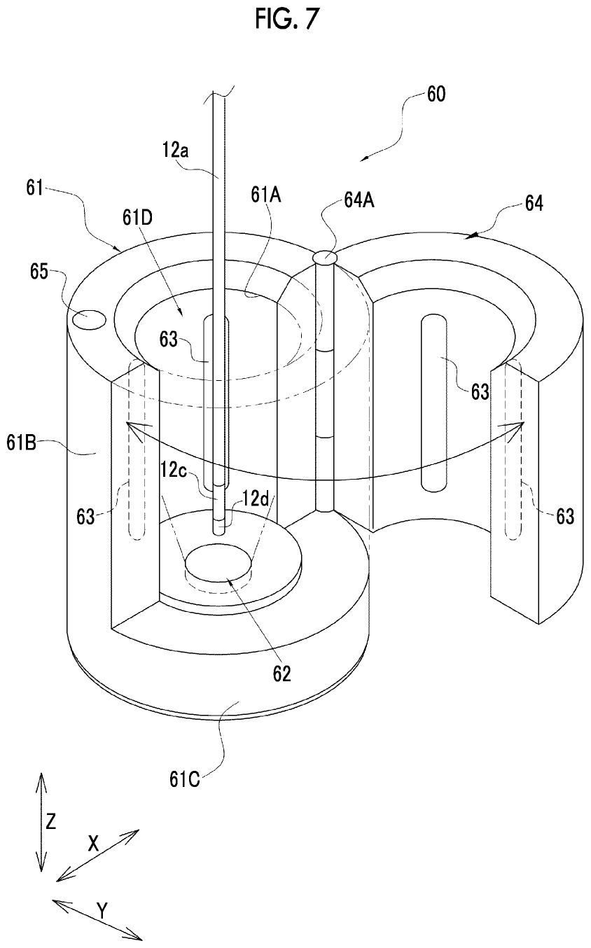

In a third embodiment which will be described below, a lid member that constitutes at least a portion of a bottom surface portion or a peripheral surface portion may be provided in addition to the configurations of the above-described first and second embodiments and the lid member may be movable such that the inside of an accommodation portion is exposed. An endoscope sterilization device 60 of the present embodiment shown in FIG. 7 may be used after being placed on the endoscope cart 20 as with the above-described first embodiment and may be used after being attached to the endoscope cart 20 as with the above-described second embodiment.

The endoscope sterilization device 60 includes a housing 61 as an accommodation portion, sterilization light sources 62 and 63, a light source control unit (not shown), a lid member 64, and a power switch 65. The endoscope sterilization device 60 sterilizes at least a portion of the insertion portion of the endoscope 12 and mainly sterilizes the distal end portion 12d. In addition, the housing 61 includes a space in which a plurality of the distal end portions 12d can be accommodated.

The housing 61 is formed in a bottomed tubular shape including an opening portion 61A, a peripheral surface portion 61B, and a bottom surface portion 61C. The peripheral surface portion 61B is formed in a cylindrical shape extending in the vertical direction Z. The bottom surface portion 61C closes a lower end of the peripheral surface portion 61B. A space inside the housing 61 that is surrounded by the peripheral surface portion 61B and the bottom surface portion 61C is a sterilization space 61D, which is a space in which irradiation with ultraviolet rays for sterilization is performed. The sterilization space 61D is formed to have dimensions larger than at least the outer diameter and the axial length of the distal end portion 12d. The opening portion 61A is a circular through-hole that is positioned in an upper surface of the peripheral surface portion 61B and through which a plurality of the soft portions 12a pass. Accordingly, the soft portions 12a can be inserted into the sterilization space 61D from the outside of the housing 61 through the opening portion 61A. The power switch 65 is provided in the vicinity of the opening portion 61A.

The sterilization light source 62 is provided on the bottom surface portion 61C of the housing 61. As the sterilization light source 62, an ultraviolet ray source is used. The sterilization light source 62 is exposed at a lowermost surface in the sterilization space 61D. The sterilization light source 62 includes an optical member (not shown) and diffuses ultraviolet rays to irradiate the sterilization space 61D with the ultraviolet rays. The ultraviolet rays can be diffused such that the range of irradiation covers at least all of the surfaces of the distal end portion 12d.

The sterilization light sources 63 are provided on the peripheral surface portion 61B of the housing 61. The sterilization light sources 63 are long sterilization light sources provided on an inner peripheral surface in the sterilization space 61D, and are disposed to be parallel with the vertical direction Z. Note that as will be described later, since the lid member 64 constitutes a portion of the peripheral surface portion 61B, a portion of a plurality of the sterilization light sources 63 is provided on an inner peripheral surface of the lid member 64. Note that as the sterilization light sources 62 and 63, the same ultraviolet ray sources as the above-described sterilization light sources 42, 43, 52, and 53 in the first embodiment are used. However, it is preferable that a flat plate-type excimer lamp is used as the sterilization light source 62 and cylindrical type excimer lamps are used as the sterilization light sources 63. The sterilization light sources 62 and 63 are controlled by the light source control unit. The light source control unit of the present embodiment performs the same control as the light source control unit 44 of the first embodiment, and the description thereof will be omitted.

The lid member 64 constitutes at least a portion of the peripheral surface portion 61B and is provided to be movable with respect to the housing 61. Specifically, the lid member 64 is connected to the housing 61 via a rotary shaft 64A such that the lid member 64 can move rotationally. The lid member 64 can move rotationally between a closing position (a position represented by two-dot chain lines) at which the sterilization space 61D of the housing 61 is covered and an opening position (a position represented by solid lines) at which the sterilization space 61D of the housing 61 is exposed. Accordingly, in addition to the effects of the first and second embodiments, in a case where the endoscope sterilization device 60 is to be cleaned, the inside of the housing 61 (that is, the sterilization space 61D) can be exposed with the lid member 64 moved to the opening position. Therefore, it is possible to easily clean the endoscope sterilization device 60.

In addition, a configuration in which the lid member 64 is attachably and detachably separated from the housing 61 may also be adopted. In this case, for example, a configuration may also be adopted in which a locking claw is provided on one of the housing 61 or the lid member 64, a locking hole is provided on the other of the housing 61 or the lid member 64, the housing 61 and the lid member 64 are integrated with each other with the locking claw and the locking hole locked, and the housing 61 and the lid member 64 are separated from each other with the locking claw and the locking hole unlocked.

Note that in the first to third embodiments, the sterilization light sources are provided on the peripheral surface portion and the bottom surface portion of the housings 41, 51, and 61, respectively. However, the present invention is not limited to this, and for example, a sterilization light source may be provided on only one of the peripheral surface portion or the bottom surface portion of the housings 41, 51, and 61 and a reflective material may be provided inside (at a position facing the sterilization spaces 41D, 51D, and 61D) the other of the peripheral surface portion or the bottom surface portion so that ultraviolet rays from the sterilization light source is reflected.

### First Modification Example

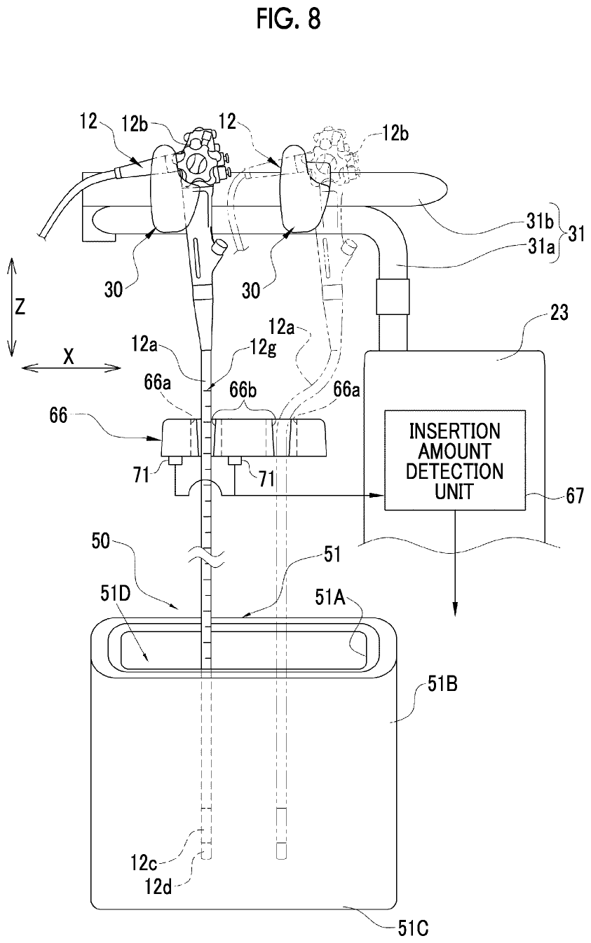

In addition, as a modification example, as shown in FIG. 8, an insertion portion holding portion 66 and an insertion amount detection unit 67 may be provided in addition to the configurations of the endoscope sterilization devices and the endoscope sterilization systems of the first to third embodiments.

The insertion portion holding portion 66 (refer to FIG. 5 as well) is provided, for example, at the endoscope cart 20. The insertion portion holding portion 66 is disposed in the vicinity of the hangers 30. Specifically, the insertion portion holding portion 66 is disposed to match the position (a position represented by solid lines in FIG. 8) of the soft portion 12a hanging down in a case where the operation portion 12b is held by the hanger 30. In the example shown in FIG. 5, the insertion portion holding portion 66 holds the soft portion 12a accommodated in the housing 51 of the endoscope sterilization device 50. At the insertion portion holding portion 66, for example, opening portions 66a of which the inner diameter matches the outer diameter of the soft portion 12a and notches 66b cut from a side surface of the insertion portion holding portion 66 toward the opening portions 66a are formed. Accordingly, at the insertion portion holding portion 66, the soft portion 12a inserted through the notch 66b is held at the opening portion 66a.

In the case of application to the example shown in FIG. 5, the insertion amount detection unit 67 detects the amount of insertion of the soft portion 12a into the housing 51 of the endoscope sterilization device 50. The insertion amount detection unit 67 has the same configuration as a well-known configuration for detection of the amount of insertion of an insertion portion into a subject. An outer peripheral surface of the soft portion 12a of the endoscope 12 is provided with measurement gradations 12g for measurement of the length of insertion of an insertion portion into a subject. The insertion portion holding portion 66 is provided with a gradations detection sensor 71 that detects the measurement gradations 12g. The gradations detection sensor 71 transmits, to the insertion amount detection unit 67, a detection signal with respect to the detected measurement gradations 12g. Since the positional relationship between the housing 51 and the insertion portion holding portion 66 is determined in advance, the insertion amount detection unit 67 detects the amount of insertion of the soft portion 12a into the housing 51 based on the detection signal of the gradations detection sensor 71.

The insertion amount detection unit 67 transmits, to the light source control unit of the endoscope sterilization device 50, the detected amount of insertion of the insertion portion of the endoscope 12, for example. The light source control unit of the endoscope sterilization device 50 controls the sterilization light sources 52 and 53 in accordance with the amount of insertion of the insertion portion that is detected by the insertion amount detection unit 67 such that the sterilization light sources 52 and 53 are controlled to start irradiation with ultraviolet rays in a case where the amount of insertion of the insertion portion into the housing 51 is equal to or larger than a certain value and the sterilization light sources 52 and 53 are controlled to stop the irradiation with the ultraviolet rays in a case where the amount of insertion of the insertion portion into the housing 51 is smaller than the certain value, for example.

Note that regarding examples shown in FIG. 5 and FIG. 8, examples of application of the insertion portion holding portion 66 and the insertion amount detection unit 67 to the endoscope sterilization device 50 and the endoscope sterilization system of the second embodiment have been described. However, the insertion portion holding portion 66 and the insertion amount detection unit 67 may be applied to the endoscope sterilization device and the endoscope sterilization system of the first and third embodiments.

In addition, in the modification example, the insertion portion holding portion 66 is disposed to match the position (the position of the soft portions 12a that is represented by the solid lines in FIG. 8) of the soft portion 12a hanging down in a case where the operation portion 12b is held by the hanger 30. Meanwhile, in a case where the position of the insertion portion holding portion 66 does not match the position of the soft portion 12a hanging down from a holding position of the hanger 30 (the position of the soft portion 12a represented by two-dot chain lines in FIG. 8), a portion of the soft portion 12a may be bent and positioned by being held at the opening portion 66a with the insertion portion holding portion 66 used as a positioning portion. Accordingly, the position of the soft portion 12a can be defined at a position where the soft portion 12a can be inserted into the accommodation portion of the endoscope sterilization device.

### Second Modification Example

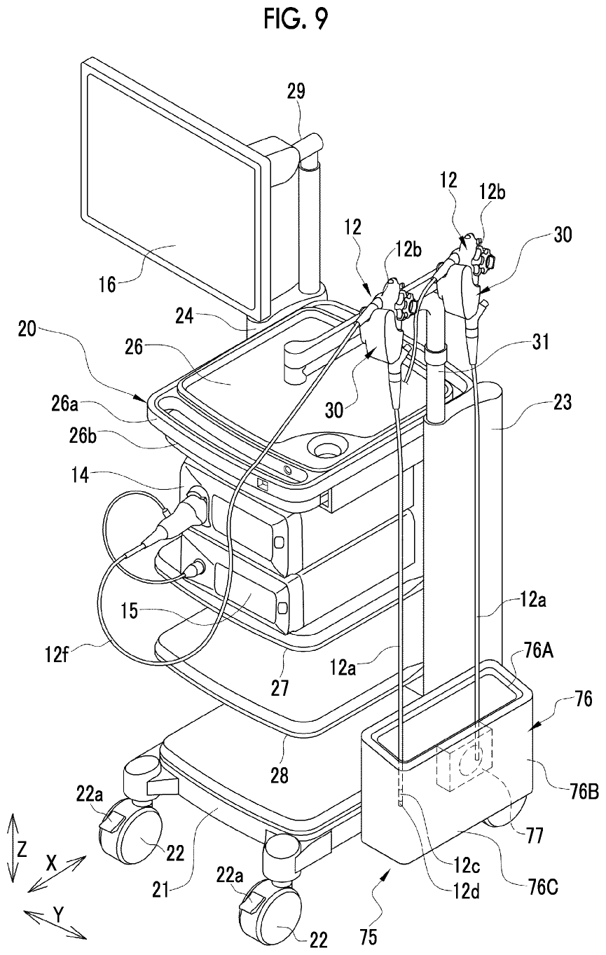

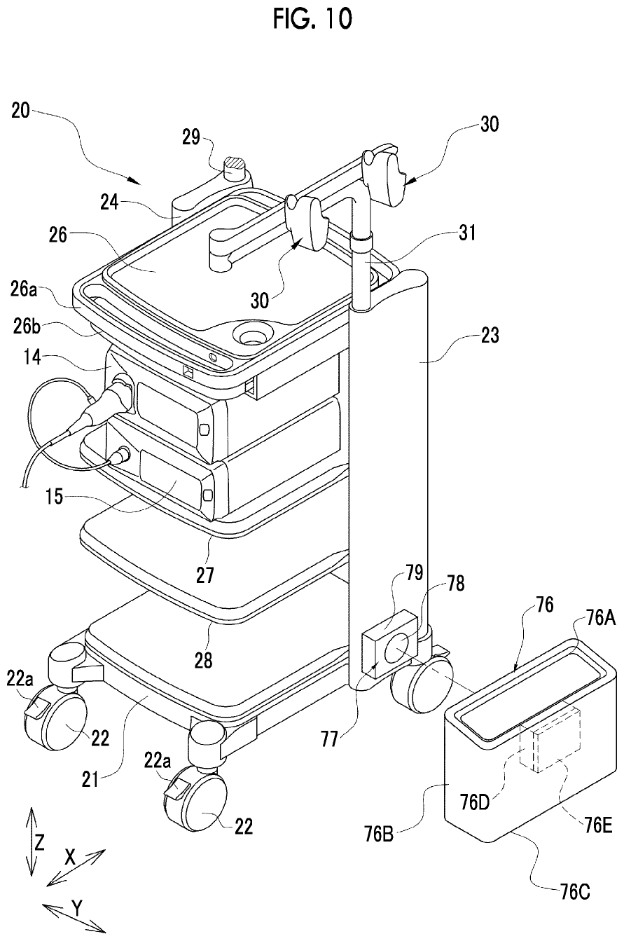

The endoscope sterilization device in each of the above-described embodiments is used separately from the endoscope cart 20 serving as an endoscope holding device or used in a state of being fixed to the endoscope cart 20 at all times. However, the present invention is not limited thereto and a portion of the endoscope sterilization device or the entire endoscope sterilization device may be attachable to and detachable from the endoscope cart 20. In an example shown in FIG. 9 and FIG. 10, an housing 76 as an accommodation portion constituting an endoscope sterilization device 75 is attachable to and detachable from the endoscope cart 20.

The endoscope sterilization device 75 is attached to the column 23 of the endoscope cart 20 as with the endoscope sterilization device 50 in the second embodiment. The endoscope sterilization device 75 includes the housing 76 and a light source unit 77. As with the housing 51 of the endoscope sterilization device 50 in the second embodiment, the housing 76 is formed in a bottomed tubular shape including an opening portion 76A, a peripheral surface portion 76B, and a bottom surface portion 76C.

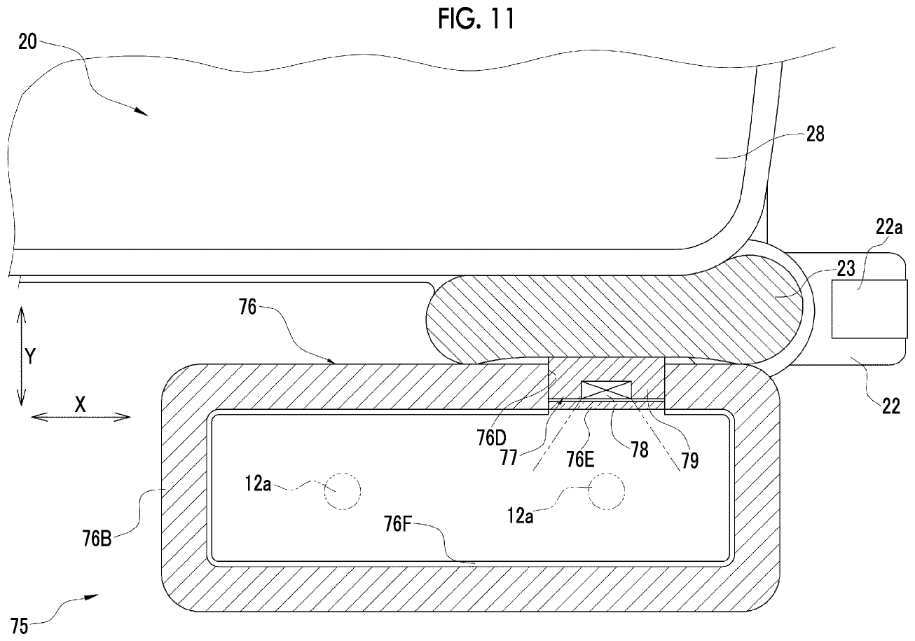

As shown in FIG. 11, in the peripheral surface portion 76B, a fitting opening portion 76D is formed at a position facing the column 23. The fitting opening portion 76D is an opening portion that has a rectangular cross section and that is cut out from the outer peripheral surface of the peripheral surface portion 76B. An inner peripheral surface of the peripheral surface portion 76B is provided with a transmission window 76E positioned to face the fitting opening portion 76D. The transmission window 76E transmits ultraviolet rays from a sterilization light source 78, which will be described later. Note that the transmission window 76E may have a function as a diffusion plate that diffuses ultraviolet rays.

In addition, the inner peripheral surface of the peripheral surface portion 76B is provided with a reflective material 76F. The reflective material 76F reflects ultraviolet rays from the sterilization light source 78, which will be described later. The configuration of the fitting opening portion 76D is not limited to the above-described configuration. The transmission window 76E may not be provided and the fitting opening portion 76D may be a through-hole that penetrates the peripheral surface portion 76B over an area from the outer peripheral surface to the inner peripheral surface.

The light source unit 77 includes the sterilization light source 78 and a fitting member 79 that holds the sterilization light source 78, and the light source unit 77 is fixed to the column 23. The fitting member 79 is formed to have a quadrangular cross section such that the outer shape thereof is fitted to the fitting opening portion 76D. The housing 76 can be attachably and detachably attached to the endoscope cart 20 with the fitting member 79 and the fitting opening portion 76D fitted to each other. In a case where the fitting opening portion 76D and the fitting member 79 are fitted to each other, the sterilization light source 78 is exposed to the inside of the housing 76 through the transmission window 76E. As the sterilization light source 78, the same ultraviolet ray source as the sterilization light sources 42 and 43 according to the first embodiment are used. Since the reflective material 76F reflects the ultraviolet rays from the sterilization light source 78, the entire region inside the housing 76 can be irradiated with the ultraviolet rays. Since the housing 76 is attachable to and detachable from the endoscope cart 20 as described above, the sterilization light source 78 is provided only on the endoscope cart 20 side. Therefore, it is possible to achieve reduction in cost. In addition, it is possible to easily clean the endoscope sterilization device 75.

Note that regarding the endoscope cart, a portion of the endoscope sterilization device is attachable to and detachable from the endoscope cart 20 in the examples shown in FIGS. 9 to 11. However, the present invention is not limited thereto and the entire endoscope sterilization device may be attachable to and detachable from the endoscope cart 20.

### Fourth Embodiment

In the first to third embodiments, the endoscope sterilization device and the endoscope sterilization system in which at least a portion of the soft portion 12a of the endoscope 12 is sterilized and the distal end portion 12d is mainly sterilized have been described as an example. However, the present invention is not limited thereto, and the target of sterilization may be the entire soft portion 12a. In a fourth embodiment which will be described below, an endoscope sterilization system will be described in which a slide mechanism is provided and an endoscope sterilization device is slid by means of the slide mechanism so that the entire soft portion 12a is sterilized.

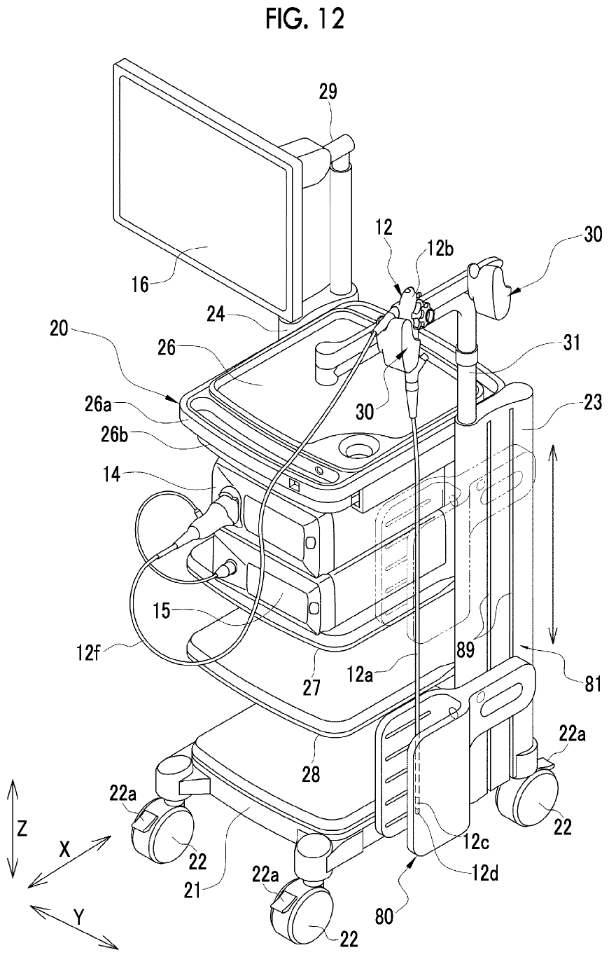

As shown in FIG. 12, an endoscope sterilization device 80 and a slide mechanism 81 are attached to the endoscope cart 20. The configuration of the endoscope cart 20 is the same as that of the first embodiment and thus the description thereof will be omitted. The endoscope sterilization device 80, the slide mechanism 81, and the endoscope cart 20 constitute an endoscope sterilization system in the scope of the claims.

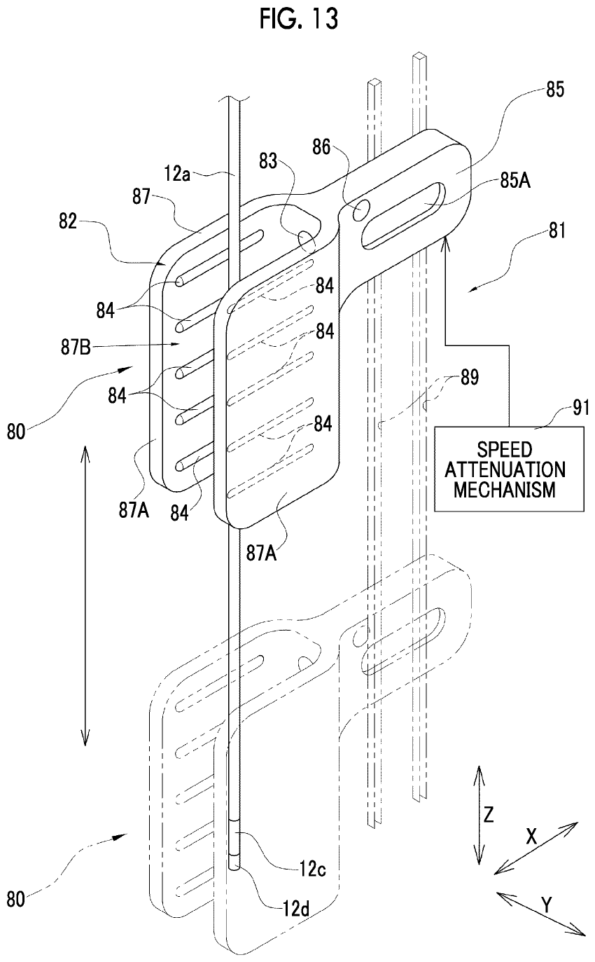

As shown in FIG. 13, the endoscope sterilization device 80 includes an insertion part 82, sterilization light sources 83 and 84, a light source control unit (not shown), an attachment piece 85, and a power switch 86. The endoscope sterilization device 80 is disposed below the hangers 30. That is, the endoscope sterilization device 80 is disposed at a position corresponding to the soft portion 12a in the hanging direction (that is, the vertical direction Z) in which the soft portion 12a hangs down because of the weight thereof in a case where the operation portion 12b is held by the hanger 30.

The insertion part 82 is formed in a U shape and includes at least a peripheral surface portion 87 that faces the soft portion 12a. As described above, since the endoscope sterilization device 80 is disposed below the hangers 30, the soft portion 12a can be inserted into the insertion part 82 in a case where the operation portion 12b of the endoscope 12 is held by the hanger 30.

In the present embodiment, linear portions 87A of the peripheral surface portion 87, which face each other, are disposed to be parallel with the front-rear direction X of the endoscope cart 20. A space inside the insertion part 82 that is surrounded by the peripheral surface portion 87 is a sterilization space 87B, which is a space in which irradiation with ultraviolet rays for sterilization is performed by the sterilization light sources 83 and 84. Accordingly, in a case where the operation portion 12b is held by the hanger 30, the sterilization light sources 83 and 84 can irradiate, with ultraviolet rays for sterilization, at least a portion of the soft portion 12a hanging down because of the weight thereof and inserted into the insertion part 82. The sterilization space 87B is formed to have dimensions larger than at least the outer diameter and the axial length of the distal end portion 12d.

As the sterilization light source 83, an ultraviolet ray source is used. One or more sterilization light sources 83 are provided with respect to the peripheral surface portion 87 and are exposed at an innermost surface in the sterilization space 87B. The sterilization light source 83 includes an optical member (not shown) and diffuses ultraviolet rays to irradiate the sterilization space 87B with the ultraviolet rays.

The sterilization light sources 84 are provided on the linear portions 87A of the peripheral surface portion 87. The sterilization light sources 84 are long sterilization light sources provided on surfaces in the sterilization space 87B that face each other, and are disposed to be parallel with the front-rear direction X. Note that as the sterilization light sources 83 and 84, the same ultraviolet ray sources as the above-described sterilization light sources 42 and 43 in the first embodiment are used. However, it is preferable that a flat plate-type excimer lamp is used as the sterilization light source 83 and cylindrical type excimer lamps are used as the sterilization light sources 84. The sterilization light sources 83 and 84 are controlled by the light source control unit. The light source control unit of the present embodiment performs the same control as the light source control unit 44 of the first embodiment, and the description thereof will be omitted.

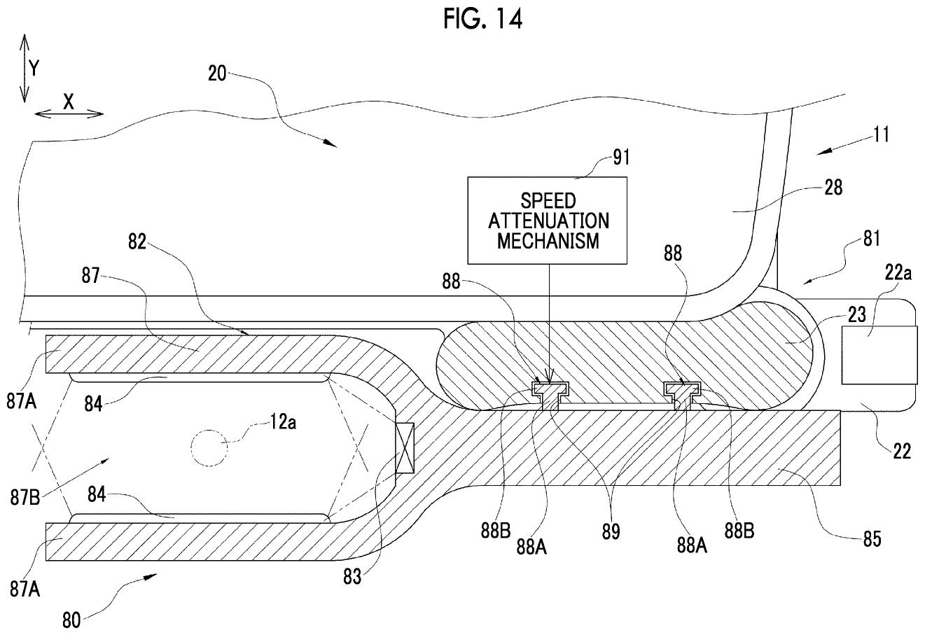

The attachment piece 85 is integrally provided with the insertion part 82. The attachment piece 85 is formed in a plate-like shape extending in the front-rear direction X. The attachment piece 85 is integrally provided with fitting members 88 (refer to FIG. 14) that constitute the slide mechanism 81. The fitting members 88 will be described later. In addition, the attachment piece 85 includes a recess portion 85A that is recessed in comparison with the surrounding area and a user can easily hook a hand on the recess portion 85A. In addition, the attachment piece 85 is provided with the power switch 86.

The slide mechanism 81 includes the fitting members 88, slide grooves 89, and a speed attenuation mechanism 91. The slide grooves 89 are linear grooves that are formed on a side surface of the column 23 and that extend in the vertical direction Z. The fitting members 88 are disposed at positions on the attachment piece 85 to face the slide grooves 89. Regarding the fitting members 88, fitting portions 88A formed to match the width of the slide grooves 89 and retaining portions 88B formed to have a width larger than the width of the slide grooves 89 are integrally formed with each other. The retaining portions 88B prevent the fitting members 88 from falling off the slide grooves 89.

As shown in FIG. 14, the fitting portions 88A are slidably fitted to the slide grooves 89. Accordingly, the endoscope sterilization device 80 is held to be slidable in the vertical direction Z with respect to the slide grooves 89. That is, the endoscope sterilization device 80 is slidable along the hanging direction in which the soft portion 12a hangs down because of the weight thereof.

The speed attenuation mechanism 91 attenuates a movement speed at which the endoscope sterilization device 80 is slid. Specifically, the speed attenuation mechanism 91 is composed of, for example, a gas spring and is disposed between the endoscope sterilization device 80 and the column 23. Note that, in FIGS. 13 and 14, the speed attenuation mechanism 91 is disposed outside the column 23 for the sake of convenience of illustration. However, the speed attenuation mechanism 91 is actually disposed at a position at which the speed attenuation mechanism 91 cannot be visually recognized from the outside (for example, the speed attenuation mechanism 91 is disposed inside the column 23).

As is well known, a gas spring is a spring that uses a reaction force based on elasticity in the case of compression of a gas sealed in a cylinder. The gas spring is attached in a state of being contracted in a contraction direction in which the sealed gas is compressed, and always generates a reaction force with respect to the endoscope sterilization device 80 in a direction opposite to the direction of a downward movement caused by the weight of the endoscope sterilization device 80 (that is, in a direction in which the endoscope sterilization device 80 is raised). Note that the configuration of the speed attenuation mechanism 91 is not limited to the above-described configuration as long as the speed attenuation mechanism 91 attenuates the speed of the sliding movement of the endoscope sterilization device 80. For example, a member having a high frictional resistance may be used for any of the slide grooves 89 or the fitting members 88 and the speed attenuation mechanism 91 may attenuate the speed of the sliding movement of the endoscope sterilization device 80 by means of a frictional force generated between the slide grooves 89 and the fitting members 88.

The action of the above-described configuration will be described. In a case where the operation portion 12b of the endoscope 12 is held by the hanger 30 before observation is performed by means of the endoscope 12, the soft portion 12a can be inserted into the insertion part 82 as described above. In addition, a doctor or a medical staff, who is a user, operates the power switch 86 of the endoscope sterilization device 80 to turn on the power. In addition, the sterilization light sources 83 and 84 can irradiate, with ultraviolet rays for sterilization, at least a portion of the soft portion 12a inserted into the insertion part 82. The doctor or the medical staff holds the endoscope sterilization device 80 and releases the endoscope sterilization device 80 after sliding the endoscope sterilization device 80 up to upper ends of the slide grooves 89, so that the endoscope sterilization device is gradually lowered because of the weight thereof. In a case where the endoscope sterilization device 80 is slid, the soft portion 12a is irradiated with ultraviolet rays for sterilization by the sterilization light sources 83 and 84, so that the entire soft portion 12a can be sterilized. The entire soft portion 12a is reliably sterilized by being irradiated with the ultraviolet rays, and thus a high sterilization effect can be achieved.

Note that in the fourth embodiment, a slide mechanism with which a user manually slides the endoscope sterilization device 80 has been described as an example of the slide mechanism 81. However, the present invention is not limited thereto and for example, a slide mechanism that includes a motor as a drive source and a transmission mechanism converting a rotational motion of the motor to a linear motion and that automatically slides the endoscope sterilization device 80 may also be used instead of the above-described configuration.

### Fifth Embodiment

In the fourth embodiment described above, the endoscope sterilization system in which the endoscope sterilization device is slid by means of the slide mechanism so that the entire soft portion 12a of the endoscope 12 is sterilized has been described as an example. However, the present invention is not limited thereto and a user may grip the endoscope sterilization device to sterilize the entire soft portion 12a of the endoscope 12. In a fifth embodiment which will be described below, an endoscope sterilization device integrally provided with a grip portion to be gripped by a user and an endoscope sterilization system will be described.

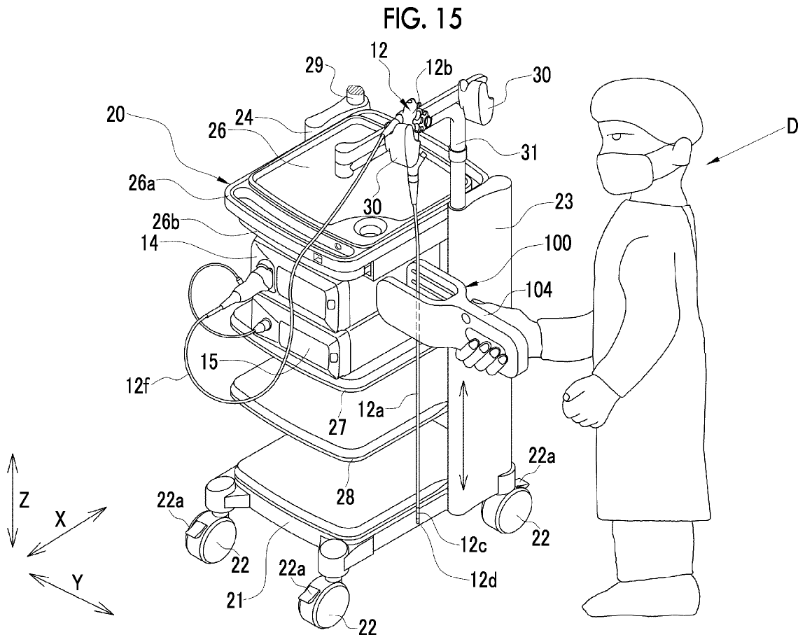

As shown in FIG. 15, an endoscope sterilization device 100 is used together with the endoscope cart 20. In addition, in FIG. 15, the display 16 is not shown in order to prevent complication. The endoscope sterilization device 100 and the endoscope cart 20 constitute an endoscope sterilization system in the scope of the claims. The configuration of the endoscope cart 20 is the same as that of the first embodiment and thus the description thereof will be omitted.

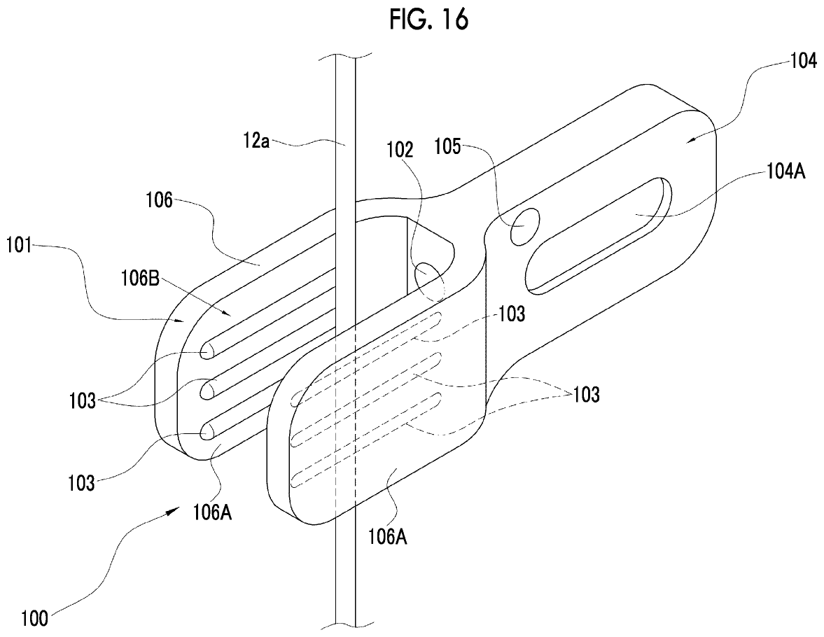

As shown in FIG. 16, the endoscope sterilization device 100 includes an insertion part 101, sterilization light sources 102 and 103, a light source control unit (not shown), a grip portion 104, and a power switch 105. The insertion part 101 is formed in a U shape and includes at least a peripheral surface portion 106 that faces the soft portion 12a. In a case where the operation portion 12b of the endoscope 12 is held by the hanger 30 and the insertion part 101 is disposed in the vicinity of the soft portion 12a, the soft portion 12a can be inserted into the insertion part 101.

The peripheral surface portion 106 includes linear portions 106A that face each other. A space inside the insertion part 101 that is surrounded by the peripheral surface portion 106 is a sterilization space 106B, which is a space in which irradiation with ultraviolet rays for sterilization is performed by the sterilization light sources 102 and 103. Accordingly, in a case where the operation portion 12b is held by the hanger 30 and the insertion part 101 is disposed in the vicinity (a position facing the outer peripheral surface of the soft portion 12a) of the soft portion 12a, the sterilization light sources 102 and 103 can irradiate, with ultraviolet rays for sterilization, at least a portion of the soft portion 12a hanging down because of the weight thereof and inserted into the insertion part 101. The sterilization space 106B is formed to have dimensions larger than at least the outer diameter and the axial length of the distal end portion 12d.

As the sterilization light source 102, an ultraviolet ray source is used. One or more sterilization light sources 102 are provided with respect to the peripheral surface portion 106 and are exposed at an innermost surface in the sterilization space 106B. The sterilization light source 102 includes an optical member (not shown) and diffuses ultraviolet rays to irradiate the sterilization space 106B with the ultraviolet rays.

The sterilization light sources 103 are provided on the linear portions 106A of the peripheral surface portion 106. The sterilization light sources 103 are long sterilization light sources provided on surfaces in the sterilization space 106B that face each other. Note that as the sterilization light sources 102 and 103, the same ultraviolet ray sources as the above-described sterilization light sources 42 and 43 in the first embodiment are used. However, it is preferable that a flat plate-type excimer lamp is used as the sterilization light source 102 and cylindrical type excimer lamps are used as the sterilization light sources 103. The sterilization light sources 102 and 103 are controlled by the light source control unit. The light source control unit of the present embodiment performs the same control as the light source control unit 44 of the first embodiment, and the description thereof will be omitted.

The grip portion 104 is integrally provided with the insertion part 82. The grip portion 104 is formed in a plate-like shape. The grip portion 104 is formed to have a size and a thickness to be easily gripped by a hand of an ordinary person. In addition, the grip portion 104 includes a recess portion 104A that is recessed in comparison with the surrounding area and a user can easily hook a hand on the recess portion 104A. In addition, the grip portion 104 is provided with the power switch 105.

The action of the above-described configuration will be described. Before observation is performed by means of the endoscope 12, the operation portion 12b of the endoscope 12 is held by the hanger 30. A doctor or a medical staff, who is a user, holds the grip portion 104 (held by a doctor D in FIG. 15) of the endoscope sterilization device 100, and the soft portion 12a can be inserted into the insertion part 101. Next, the power switch 105 of the endoscope sterilization device 100 is operated such that the power is turned on. In addition, the sterilization light sources 102 and 103 can irradiate, with ultraviolet rays for sterilization, at least a portion of the soft portion 12a inserted into the insertion part 101. While maintaining such a state, the doctor or the medical staff holds the grip portion 104 of the endoscope sterilization device 100 and gradually lowers the endoscope sterilization device 100 to a distal end of the soft portion 12a to a proximal end or raises the endoscope sterilization device 100 to the proximal end from the distal end, for example. In a case where the endoscope sterilization device 100 is manually moved, the soft portion 12a is irradiated with ultraviolet rays for sterilization by the sterilization light sources 102 and 103, so that the entire soft portion 12a can be sterilized. The entire soft portion 12a is reliably sterilized by being irradiated with the ultraviolet rays, and thus a high sterilization effect can be achieved.

### Sixth Embodiment

In the fifth embodiment described above, the endoscope sterilization device that is manually moved and used only for sterilization of the entire soft portion 12a has been described as an example. However, the present invention is not limited thereto and an endoscope sterilization device that can switch between a mode in which the distal end portion 12d is inserted to be sterilized and a mode in which the endoscope sterilization device is manually moved so that the entire soft portion 12a is sterilized. In a sixth embodiment which will be described below, an endoscope sterilization device and an endoscope sterilization system, which can switch to a mode in which the distal end portion 12d is inserted to be sterilized in the case of an installation state and can switch to a mode in which the endoscope sterilization device is manually moved so that the entire soft portion 12a is sterilized in the case of a state of being gripped by a user, will be described.

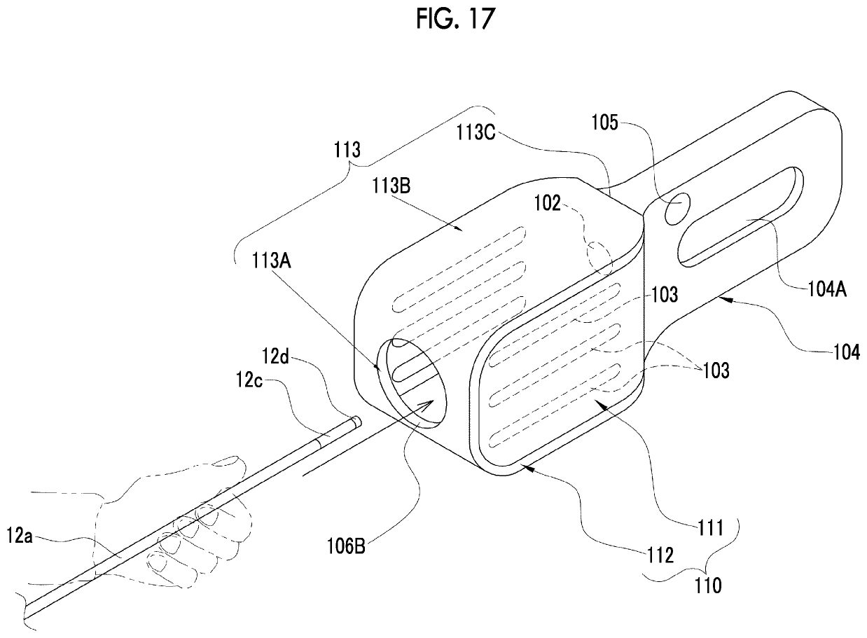

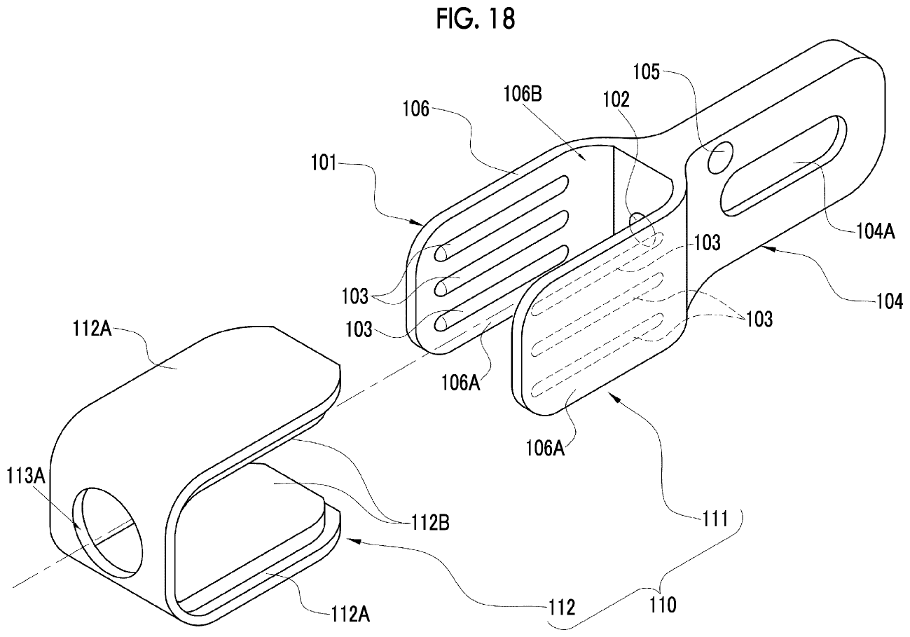

As shown in FIGS. 17 and 18, an endoscope sterilization device 110 includes a sterilization device main body 111 and a cover member 112. Since the sterilization device main body 111 has the same configuration as the endoscope sterilization device 100 according to the fifth embodiment and includes the insertion part 101, the sterilization light sources 102 and 103, the grip portion 104, the light source control unit, and the power switch 105, the same reference numerals are given thereto and the description thereof will be omitted. The endoscope sterilization device 110 is used together with the endoscope cart 20. The endoscope sterilization device 110 and the endoscope cart 20 constitute an endoscope sterilization system in the scope of the claims. The configuration of the endoscope cart 20 is the same as that of the first embodiment and thus the description thereof will be omitted.

The cover member 112 is formed in a U-like shape corresponding to the peripheral surface portion 106 of the sterilization device main body 111, and an open portion of one cover member or the peripheral surface portion 106 is covered by the other cover member or the peripheral surface portion 106 so that an accommodation portion 113 (refer to FIG. 14) is configured. On inner surfaces of linear portions 112A of the cover member 112 that face each other, protruding portions 112B that protrude in comparison with the inner surfaces are formed. For example, the cover member 112 and the insertion part 101 (that is, the sterilization device main body 111) can be integrated with each other with the protruding portions 112B fitted between the linear portions 106A of the peripheral surface portion 106.

The accommodation portion 113 is formed in a bottomed tubular shape including an opening portion 113A, a peripheral surface portion 113B, and a rear surface portion 113C in a case where the cover member 112 and the insertion part 101 are integrated with each other. The peripheral surface portion 113B is formed in a quadrangular tubular shape to constitute upper and lower surfaces and right and left surfaces of the accommodation portion 113. The rear surface portion 113C closes a rear end of the peripheral surface portion 113B. The rear surface portion 113C corresponds to a bottom surface portion in the scope of the claims.

A space inside the accommodation portion 113 that is surrounded by the peripheral surface portion 113B and the rear surface portion 113C is the sterilization space 106B of the insertion part 101, which is a space in which irradiation with ultraviolet rays for sterilization is performed by the sterilization light sources 102 and 103 provided in the sterilization device main body 111. The sterilization space 106B is formed to have dimensions larger than at least the outer diameter and the axial length of the distal end portion 12d, and can accommodate the distal end portion 12d. The opening portion 113A is a circular through-hole that is positioned in a front surface of the peripheral surface portion 113B and that has an inner diameter larger than at least the outer diameter of the soft portion 12a. Through the opening portion 113A, at least a portion of the soft portion 12a can be inserted into the sterilization space 106B from the outside of the accommodation portion 113.

The action of the above-described configuration will be described. In a case where the cover member 112 and the insertion part 101 are integrated with each other, the endoscope sterilization device 110 can be used as an installation type endoscope sterilization device, which is used in a state of being placed on the endoscope cart 20 as with the endoscope sterilization device 18 in the first embodiment, since the endoscope sterilization device 110 includes the accommodation portion 113 and the sterilization light sources 102 and 103. That is, before observation is performed by means of the endoscope 12, the operation portion 12b of the endoscope 12 held by the hanger 30 and then a doctor or a medical staff, who is a user, operates the power switch 105 of the endoscope sterilization device 110 to turn on the power. Then, the distal end portion 12d is inserted into the accommodation portion 113 (that is, the sterilization space 106B) through the opening portion 113A. At least a portion of the soft portion 12a including the distal end portion 12d is irradiated with ultraviolet rays from the sterilization light sources 102 and 103. The distal end portion 12d is reliably sterilized by being irradiated with the ultraviolet rays, and thus a high sterilization effect can be achieved.

Meanwhile, in a case where the cover member 112 and the insertion part 101 are separated from each other, since the sterilization device main body 111 has the same configuration as the endoscope sterilization device 100 of the fifth embodiment, the doctor or the medical staff holds the grip portion 104 of the sterilization device main body 111 and in a case where the sterilization device main body 111 is manually moved, the soft portion 12a is irradiated with ultraviolet rays for sterilization by the sterilization light sources 102 and 103, so that the entire soft portion 12a can be sterilized.

As described above, with a simple operation of attaching and detaching the cover member 112, it is possible to switch between the mode in which the distal end portion 12d is inserted to be sterilized and the mode in which the endoscope sterilization device is manually moved so that the entire soft portion 12a is sterilized.

Note that in the fourth to sixth embodiments, the insertion parts 82 and 101 are provided with the plurality of sterilization light sources. However, the present invention is not limited thereto and a reflective material may be provided instead of a portion of the plurality of sterilization light sources so that the ultraviolet rays from the sterilization light sources are reflected by the reflective material. In addition, in the sixth embodiment, a reflective material may be provided inside the cover member 112 (at a position facing the sterilization space 106B).

### Third Modification Example

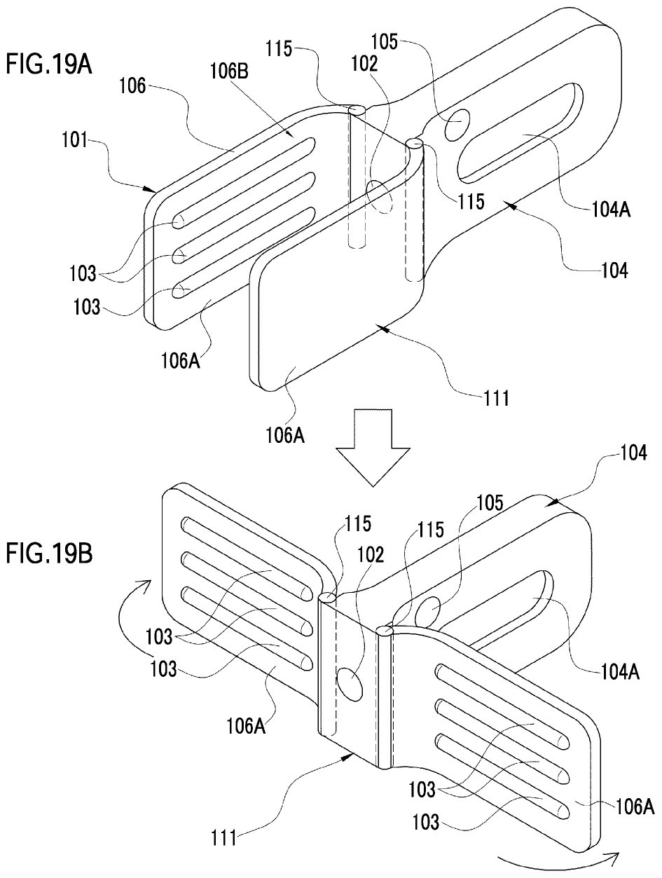

In a modification example which will be described below, in addition to the configurations of the fifth and sixth embodiments, a switch to a mode used for sterilization of an instrument for an endoscope can be made. As shown in FIG. 19A, the insertion part 101 is provided with rotary shafts 115 that rotates the linear portions 106A outward by 90 degrees.

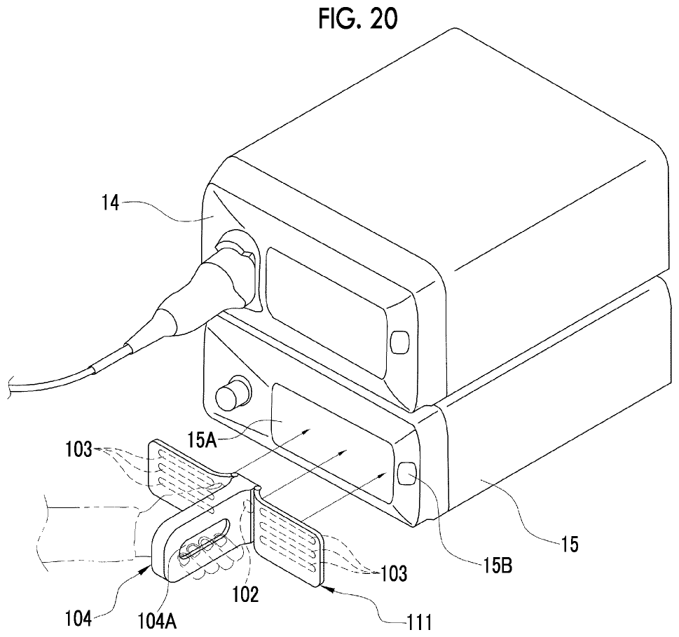

As shown in FIG. 19B, in a case where the linear portions 106A are rotated outward by 90 degrees, the linear portions 106A facing each other form a line so that all of the sterilization light sources 102 and 103 are arranged on one surface side. Accordingly, the sterilization device main body 111 can also be used for sterilization of an instrument for an endoscope such as the light source device 14 and the processor device 15, of which the surface area is larger than that of the soft portion 12a of the endoscope 12. FIG. 20 shows an example of a case where the sterilization device main body 111 of the present modification example is used for sterilization of the processor device 15 and the user can hold the grip portion 104 and cause the sterilization light sources 102 and 103 to face an operation panel 15A of the processor device 15 so that the operation panel 15A is irradiated with ultraviolet rays. Accordingly, it is possible to sterilize even a large-area object like the operation panel 15A. In addition, the present invention is not limited thereto and sterilization device main body 111 of the present modification example may also be used for sterilization of an operation switch 15B or the like.

### Seventh Embodiment

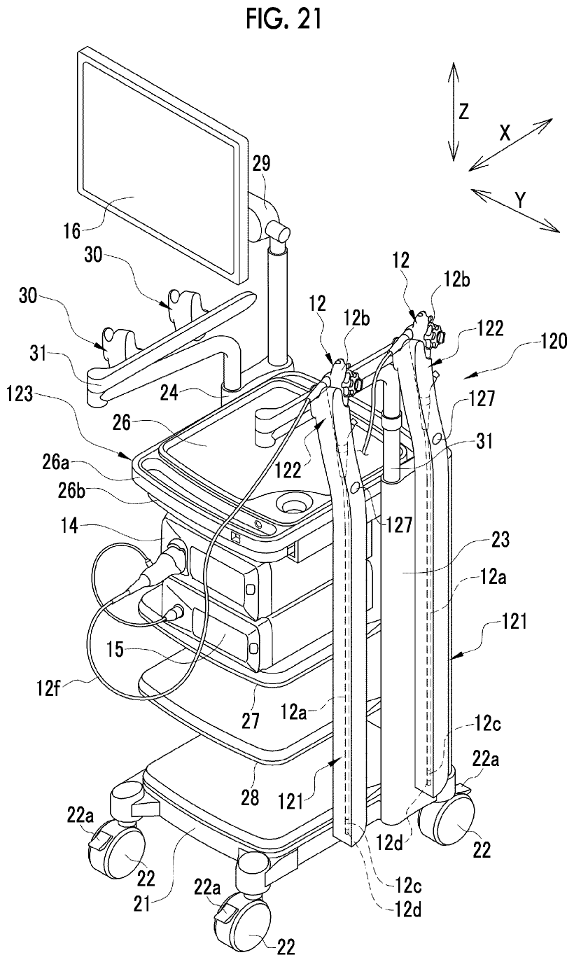

In the fourth to sixth embodiments, the endoscope sterilization device that is moved with respect to the soft portion 12a so that the entire soft portion 12a is sterilized and the endoscope sterilization system have been described as an example. However, the present invention is not limited thereto and the entire soft portion 12a may be covered so that the entire soft portion 12a is sterilized. In an endoscope sterilization system 120 of the present embodiment which is shown in FIGS. 21 to 23, hangers 122 and endoscope sterilization devices 121 that sterilize the soft portion 12a are integrally provided with each other and the entire soft portion 12a is sterilized.

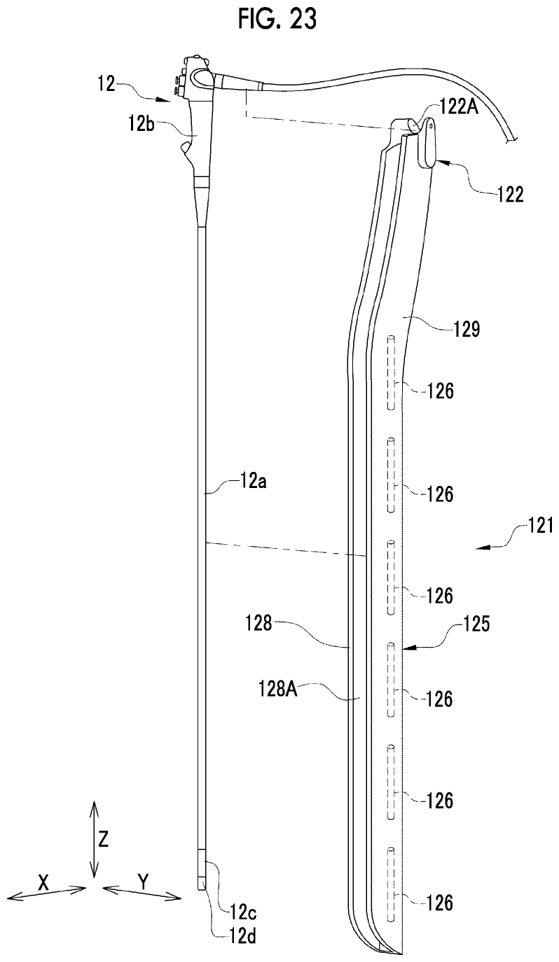

As shown in FIG. 21, the hangers 122 integrally provided with the endoscope sterilization devices 121 are attached to an endoscope cart 123 and each include a notch or a groove (in the case of an example shown in FIG. 23, a notch 122A is provided) as with the hangers 30 provided on the endoscope cart 20 in the first embodiment, and the endoscope 12 can be hung thereon with the operation portion 12b of the endoscope 12 being held at the notch or the groove. The hangers 122 are attached to the upper end of the column 23 on the right side via the hanger stand 31. In a case where the operation portion 12b is held by the hanger 122, the hanging direction in which the soft portion 12a hangs down is parallel to the vertical direction Z, the soft portion 12a hanging down because of the weight thereof.

In the present embodiment, two endoscope sterilization devices 121 and two hangers 122 are provided for one endoscope cart 123. However, the number of the endoscope sterilization devices 121 and the number of the hangers 122 are not limited to two and an appropriate number of hangers 30 are provided in accordance with the purpose of use of the endoscope 12 and the dimensions of the endoscope cart 123 and the hanger stand 31. The endoscope sterilization device 121 is positioned in the hanging direction in which the soft portion 12a hangs down because of the weight thereof in a case where the operation portion 12b is held by the hanger 122.

Note that, regarding the endoscope cart 123, the hanger 122 integrally provided with the endoscope sterilization devices 121 are attached to the column 23 on the right side and the same hangers 30 as those of the endoscope cart 20 in the first embodiment are attached to the column 24 on the left side via the hanger stand 31. Since the configuration of the endoscope cart 123 is the same as that of the endoscope cart 20 in the first embodiment in other respects, the same components will be given the same reference numerals and description thereof will be omitted.

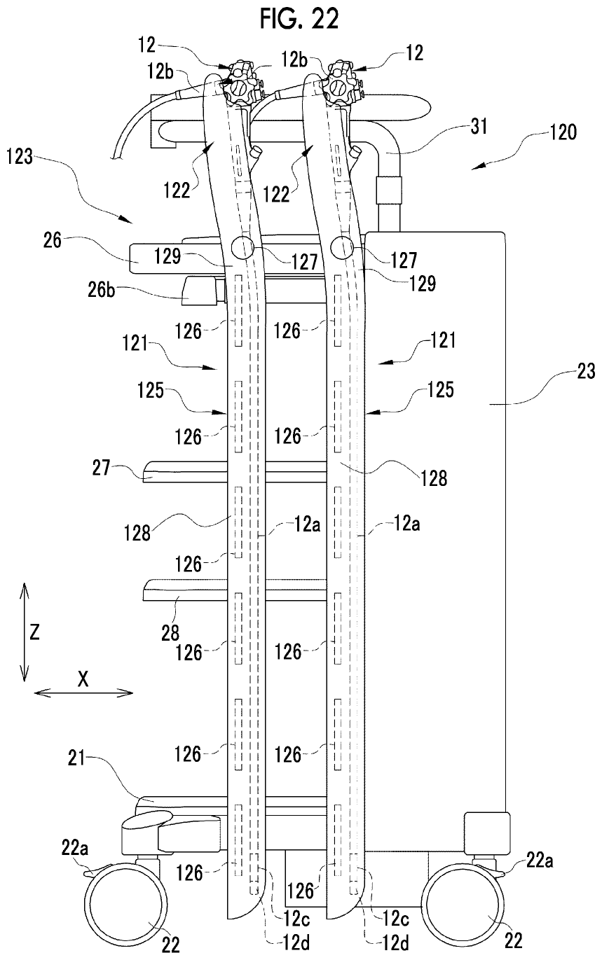

As shown in FIG. 22, the endoscope sterilization devices 121 each include an accommodation portion 125, sterilization light sources 126, and a power switch 127. The accommodation portion 125 includes at least a peripheral surface portion 128 extending along the vertical direction Z, and accommodates the soft portion 12a of the endoscope 12. In addition, the endoscope sterilization devices 121 each include a bent portion 129 that smoothly connects the hanger 122 and the peripheral surface portion 128 to each other. The power switch 127 is provided on, for example, a side surface of the bent portion 129.

The peripheral surface portion 128 has a gutter-like shape including a groove 128A (see FIG. 23) formed along the vertical direction Z, and in a case where the operation portion 12b is held by the hanger 122, the peripheral surface portion 128 faces the outer peripheral surface of the soft portion 12a hanging down because of the weight thereof.

The sterilization light sources 126 are disposed inside the groove 128A of the peripheral surface portion 128 along the vertical direction Z. The sterilization light sources 126 irradiate the soft portion 12a with light for sterilization. In addition, although a plurality of the sterilization light sources 126 are provided for one peripheral surface portion 128 in FIG. 22 and FIG. 23, the present invention is not limited thereto and one long sterilization light source 126 may be provided for one peripheral surface portion 128. Note that as the sterilization light sources 126, the same ultraviolet ray sources as the above-described sterilization light sources 42 and 43 in the first embodiment are used. However, it is preferable that cylindrical type excimer lamps are used as the sterilization light sources 126.

The action of the above-described configuration will be described. As shown in FIG. 23, the endoscope sterilization device 121 is positioned in the hanging direction in which the soft portion 12a hangs down because of the weight thereof as described above in a case where the operation portion 12b of the endoscope 12 is held by the hanger 122 before observation is performed by means of the endoscope 12. Therefore, a doctor or a medical staff, who is a user, can easily insert the soft portion 12a into the groove 128A of the endoscope sterilization device 121. In addition, the doctor or the medical staff operates the power switch 127 of the endoscope sterilization device 121 to turn on the power. The entire soft portion 12a is reliably sterilized by being irradiated with the ultraviolet rays, and thus a high sterilization effect can be achieved.

In addition, in the case of the endoscope 12 after being used, the operation portion 12b can be held by the hanger 30 attached to the column 24 on the left side. Since the hangers 30 are provided at positions separated from the endoscope sterilization devices 121 provided on the column 23 on the right side, dirt such as a body fluid does not adhere to the endoscope sterilization devices 121 and thus the endoscope sterilization devices 121 can be kept clean.

### Fourth Modification Example

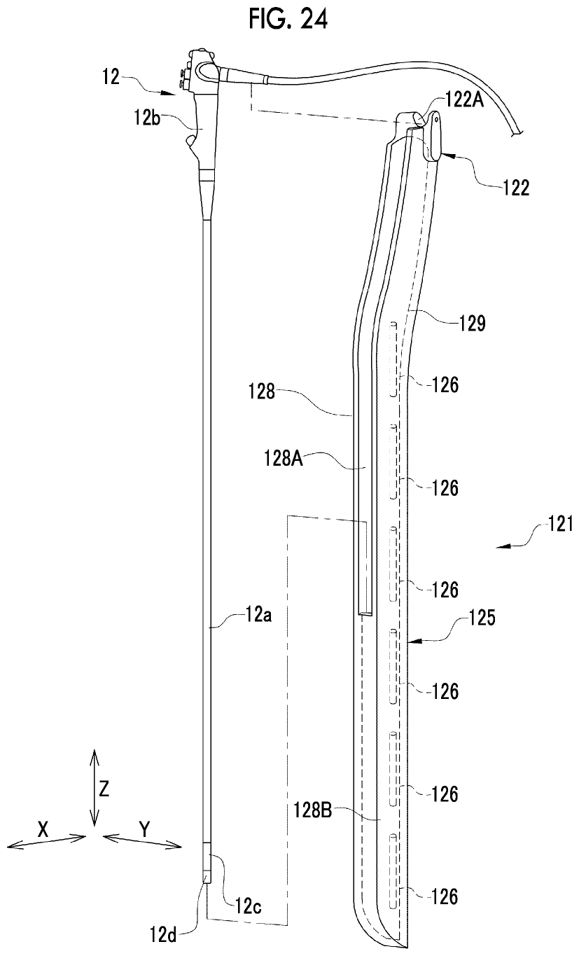

In the seventh embodiment, the entire endoscope sterilization device 121 is formed in a gutter-like shape. However, the present invention is not limited thereto and the endoscope sterilization device 121 may be formed to have a shape in which an upper portion has a gutter-like shape and a lower portion has a tubular shape as shown in FIG. 24. Specifically, a lower portion 128B (a portion in which at least the distal end portion 12d is accommodated) of the peripheral surface portion 128 of the endoscope sterilization device 121 is formed in a tubular shape having a quadrangular cross section and the soft portion 12a can be inserted thereinto. Accordingly, the outer peripheral surface of the soft portion 12a can be covered and thus the sterilization effect can be further improved. The shape of the lower portion 128B is not limited to a tubular shape having a quadrangular cross section, and may be any shape such as a cylindrical shape as long as the soft portion 12a can be inserted into the lower portion 128B.

In each of the above-described embodiments or the modification examples thereof, the endoscope cart has been described as an example of an endoscope holding device. However, the present invention is not limited thereto and the endoscope holding device may be an endoscope stand provided with a hanger holding an operation portion of an endoscope. In addition, as the reflective material provided inside the accommodation portion in each of the above-described embodiments or the modification examples thereof, for example, a metal sheet of which a surface is formed to have a mirror-like shape, or a member of which a surface is plated is used.

In addition, although the endoscope 12 to be combined with the endoscope sterilization device or the endoscope sterilization system according to the embodiments of the present invention has not been specified in the above-described embodiments, the endoscope 12 may be any endoscope as long as an insertion portion is provided and may be a bronchoscope, an upper gastrointestinal tract endoscope, or a lower gastrointestinal tract endoscope, for example.

In each of the above-described embodiments, a hardware structure of a processing unit that performs various types of processing, such as the light source control unit, is various processors as described below. The various processors include a central processing unit (CPU), which is a general-purpose processor that executes software (a program) and functions as various processing units, a graphical processing unit (GPU), a programmable logic device (PLD) such as a field programmable gate array (FPGA), which is a processor of which the circuit configuration can be changed after being manufactured, and a dedicated electric circuit which is a processor having a circuit configuration exclusively designed for various processes.

One processing unit may be composed of one of these various processors or a combination of two or more processors of the same type or different types (for example, a combination of a plurality of FPGAs, a combination of a CPU and an FPGA, a combination of a CPU and a GPU, or the like). In addition, a plurality of processing units may be composed of one processor. As examples of a configuration in which a plurality of processing units are composed of one processor, firstly, there is a configuration in which one processor is composed of a combination of one or more CPUs and software and the processor functions as a plurality of processing units as represented by a computer such as a client, a server, or the like. Secondly, as represented by a system-on-chip (SoC), there is a configuration in which a processor that realizes, with one integrated circuit (IC) chip, the functions of the entire system including a plurality of processing units is used. As described above, various processing units are configured by using one or more of the above-described various processors as the hardware structure.

Furthermore, the hardware structure of the various processors is, more specifically, an electric circuit (circuitry) in which circuit elements such as semiconductor elements are combined with each other.

### EXPLANATION OF REFERENCES

10: endoscope system
12: endoscope
12a: insertion portion
12b: operation portion
12c: bendable portion
12d: distal end portion
12e: angle knob
12f: cord
12g: measurement gradation
14: light source device
15: processor device
15A: operation panel
15B: operation switch
16: display
18: endoscope sterilization device
20: endoscope cart
21: frame pedestal portion
22: caster
22a: stop lever
23, 24: column
26: top plate
26a: handle
26b: drawer portion
27, 28: shelf plate
29: stand
30: hanger
31: hanger stand
31a, 31b: stand member
41: housing
41A: opening portion
41B: peripheral surface portion
41C: rear surface portion
41D: sterilization space
42, 43: sterilization light source
44: light source control unit
45: power switch
50: endoscope sterilization device
51: housing
51A: opening portion
51B: peripheral surface portion
51C: bottom surface portion
51D: sterilization space
52, 53: sterilization light source
54: power switch
60: endoscope sterilization device
61: housing
61A: opening portion
61B: peripheral surface portion
61C: bottom surface portion
61D: sterilization space
62, 63: sterilization light source
64: lid member
65: power switch
64A: rotary shaft
66: insertion portion holding portion
66a: opening portion
66b: notch
67: insertion amount detection unit
71: gradations detection sensor
75: endoscope sterilization device
76: housing
76A: opening portion
76B: peripheral surface portion
76C: bottom surface portion
76D: fitting hole
76E: reflective material
77: light source unit
78: sterilization light source
79: fitting member
80: endoscope sterilization device
81: slide mechanism
82: insertion part
83: sterilization light source
84: sterilization light source
85: attachment piece
85A: recess portion
86: power switch
87: peripheral surface portion
87A: linear portion
87B: sterilization space
88: fitting member
88A: fitting portion
88B: retaining portion
89: slide groove
91: speed attenuation mechanism
100: endoscope sterilization device
101: insertion part
102, 103: sterilization light source
104: grip portion
104A: recess portion
105: power switch
106: peripheral surface portion
106A: linear portion
106B: sterilization space
110: endoscope sterilization device
111: sterilization device main body
112: cover member
112A: linear portion
112B: protruding portion
113: accommodation portion
113A: opening portion
113B: peripheral surface portion
113C: rear surface portion
115: rotary shaft
120: endoscope sterilization system
121: endoscope sterilization device
122: hanger
122A: notch
123: endoscope cart
125: accommodation portion
126: sterilization light source
127: power switch
128: peripheral surface portion
128A: groove
128B: lower portion
129: bent portion
CL: central axis
D: doctor
X: front-rear direction
Y: lateral direction
Z: vertical direction

What is claimed is:

1. An endoscope sterilization device which irradiates an insertion portion of an endoscope with light for sterilization, the endoscope sterilization device comprising:
an accommodation portion that has a bottomed tubular shape including an opening portion, a peripheral surface portion, and a bottom surface portion and into which at least a portion of the insertion portion is insertable through the opening portion; and
a sterilization light source that is provided in the accommodation portion and that irradiates the insertion portion inserted into the accommodation portion with the light for sterilization.

2. The endoscope sterilization device according to claim 1,
wherein the sterilization light source is provided on the bottom surface portion.

3. The endoscope sterilization device according to claim 1,
wherein the sterilization light source is provided on the peripheral surface portion.

4. The endoscope sterilization device according to claim 1,
wherein a reflective material is provided on at least one of the bottom surface portion or the peripheral surface portion.

5. The endoscope sterilization device according to claim 1,
wherein the light with which the sterilization light source performs the irradiation is ultraviolet rays of which a central wavelength is 222 nm.

6. The endoscope sterilization device according to claim 5,
wherein the sterilization light source is an excimer lamp or an LED.

7. The endoscope sterilization device according to claim 1, further comprising:
a lid member that constitutes at least a portion of the bottom surface portion or the peripheral surface portion,
wherein the lid member is movable between a closing position at which an inside of the accommodation portion is covered and an opening position at which the inside of the accommodation portion is exposed.

8. The endoscope sterilization device according to claim 7,
wherein the lid member is attachably and detachably separated from the accommodation portion.

9. The endoscope sterilization device according to claim 1, further comprising:
an insertion amount detection unit that detects an amount of insertion of the insertion portion into the accommodation portion.

10. The endoscope sterilization device according to claim 1, further comprising:
an insertion portion holding portion that holds the insertion portion inserted into the accommodation portion.

11. An endoscope sterilization system in which an insertion portion of an endoscope is irradiated with light for sterilization, the endoscope sterilization system comprising:
an endoscope holding device that is provided with a hanger holding an operation portion of the endoscope, the operation portion being consecutively connected to the insertion portion; and
the endoscope sterilization device according to claim 1.

12. The endoscope sterilization system according to claim 11,
wherein the endoscope holding device is an endoscope stand or an endoscope cart.

13. The endoscope sterilization system according to claim 11,
wherein the opening portion is open in a direction intersecting a hanging direction in which the insertion portion hangs down because of a weight thereof in a case where the operation portion is held by the hanger.

14. The endoscope sterilization system according to claim 11, further comprising:
an attachment and detachment mechanism that makes the endoscope sterilization device and the endoscope holding device attachable to each other and detachable from each other.

15. An endoscope sterilization system in which an insertion portion of an endoscope is irradiated with light for sterilization, the endoscope sterilization system comprising:
an endoscope holding device that is provided with a hanger holding an operation portion of the endoscope, the operation portion being consecutively connected to the insertion portion; and
an endoscope sterilization device that includes a gutter-shaped accommodation portion and a sterilization light source accommodated in the accommodation portion, the accommodation portion being composed of at least a peripheral surface portion that faces an outer peripheral surface of the insertion portion hanging down because of a weight thereof in a case where the operation portion is held by the hanger, and the sterilization light source irradiating the insertion portion accommodated in the accommodation portion with the light for sterilization.

16. The endoscope sterilization system according to claim 15,
wherein the light with which the sterilization light source performs the irradiation is ultraviolet rays of which a central wavelength is 222 nm.

17. The endoscope sterilization system according to claim 16,
wherein the sterilization light source is an excimer lamp or an LED.

18. An endoscope sterilization device which irradiates an insertion portion of an endoscope with light for sterilization, the endoscope sterilization device comprising:
an insertion part that includes a peripheral surface portion facing the insertion portion and into which the insertion portion is insertable; and
a sterilization light source that irradiates the insertion portion inserted into the insertion part with the light for sterilization.

19. The endoscope sterilization device according to claim 18,
wherein the light with which the sterilization light source performs the irradiation is ultraviolet rays of which a central wavelength is 222 nm.

20. The endoscope sterilization device according to claim 19,
wherein the sterilization light source is an excimer lamp or an LED.

21. An endoscope sterilization system in which an insertion portion of an endoscope is irradiated with light for sterilization, the endoscope sterilization system comprising:
an endoscope holding device that is provided with a hanger holding an operation portion of the endoscope, the operation portion being consecutively connected to the insertion portion;
the endoscope sterilization device according to claim 18; and
a slide mechanism that holds the endoscope sterilization device to be slidable along a hanging direction in which the insertion portion hangs down because of a weight thereof in a case where the operation portion is held by the hanger.

22. The endoscope sterilization system according to claim 21,
wherein the slide mechanism includes a speed attenuation mechanism that attenuates a speed of movement of the endoscope sterilization device.

23. The endoscope sterilization system according to claim 21,
wherein the endoscope holding device is an endoscope stand or an endoscope cart.

24. The endoscope sterilization device according to claim 18, further comprising:
a grip portion that is integrally provided with the insertion part.

25. The endoscope sterilization device according to claim 24, further comprising:
a cover member that is provided to be attachable to and detachable from the insertion part,
wherein, in a case where the cover member and the insertion part are integrated with each other, the cover member and the insertion part constitute an accommodation portion that has a bottomed tubular shape including an opening portion, a peripheral surface portion, and a bottom surface portion and into which at least a portion of the insertion portion is insertable through the opening portion.
